(19) United States
(12) Patent Application Publication
(10) Pub. No.: US 2026/0207204 A1
(43) Pub. Date: Jul. 23, 2026

(54) TIGHTENING MECHANISM

(71) Applicant: XAVIER TECHNOLOGIES, Garden Grove, CA (US)

(72) Inventors: Johnny CARABAJAL, Garden Grove, CA (US); James PEÑA, Vista, CA (US)

(21) Appl. No.: 19/142,494

(22) PCT Filed: Dec. 28, 2023
(86) PCT No.: PCT/US2023/086325

§ 371 Date: Jun. 23, 2025

Related U.S. Application Data
(60) Provisional application No. 63/435,678, filed on Dec. 28, 2022.

(51) Int. Cl. A61B 17/132

(52) U.S. Cl. CPC A61B 17/1327

(57) ABSTRACT

A tightening mechanism includes a spool being configured to accept a lace. The tightening mechanism includes a ratchet wheel operably connected to the spool. The tightening mechanism includes a knob operably connected to the ratchet wheel, the knob being configured for manually winding a portion of the lace onto the spool in a winding direction. The tightening mechanism includes a first pawl arm including a first pawl, the first pawl being configured to engage with the ratchet wheel to prevent the spool from moving in a direction opposite to the winding direction. The tightening mechanism includes a second pawl arm including a second pawl, the second pawl being configured to engage with the ratchet wheel to prevent the spool from moving in the direction opposite to the winding direction. The tightening mechanism includes a spring being configured to bias the first pawl and the second pawl towards the ratchet wheel.

### BACKGROUND

Hemorrhage is the leading cause of combat deaths. In many cases, applying a tourniquet after sustaining a wound in an appendage is too painful to apply effectively. In some cases, a person, such as an injured soldier, law enforcement officer, or government agent loses consciousness and is unable to apply self-aid. Self-aid on multiple extremities is unlikely in most battleground cases since each solder is typically issued one tourniquet. In many cases, medical personnel cannot reach a wounded soldier, law enforcement officer, or government agent in time to locate a tourniquet, place the tourniquet in position, secure the tourniquet on a limb, tighten the tourniquet, and (optionally) lock the tourniquet in a tightened position to cease a hemorrhage.

Many existing tourniquets may not be considered as effective devices to occlude blood flow to prevent spread of venom throughout the body. This is due to the amount of time it typically takes to don and effectively activate many of the existing tourniquets. Venom may be secreted by invertebrates, fish, amphibians, and reptiles. Examples of animals that may secrete venom include stonefish, stingrays, jellyfish, bees, wasps, spiders, komodo dragons, snakes, and scorpions.

Many existing tourniquets may have excess weight and/or bulk. Excess weight and/or bulk may reduce the number of tourniquets carried by a user or a first responder.

Many existing tourniquets may be difficult to place on a limb for effective occlusion. Many existing tourniquets may be difficult to operate. Many existing tourniquets may not be adaptable to a variety of limb sizes and limb types (for example, legs and arms). Operation by a person who has not been trained in proper tourniquet placement and operation is often too difficult to complete effectively. In some cases, a person may be in too much pain to effectively apply self-aid to occlude blood flow. Operation by medical personnel may take too much time in cases involving mass injuries such as, for example, on a battlefield, in a terrorist attack, in a natural disaster, or after a multivehicle accident.

Many existing tourniquets may not provide the durability and/or the wearability necessary for effective combat and/or immediate use. Many existing tourniquets may be difficult to operate while wearing gloves. Many existing tourniquets may be difficult to operate with wet hands. Many existing tourniquets may not apply enough pressure to completely occlude blood flow in an artery of a wearer. Many existing tourniquets may rely on a pressure ridge that needs to be located directly over an artery to occlude blood flow. Many existing tourniquets may cause scrunching of tourniquet material during use which may cause unnecessary discomfort to the wearer and/or may prevent effective occlusion. Many existing tourniquets may locate an adjustment mechanism on a medial side of a limb. In this location, the adjustment mechanism may be engaged or disengaged prematurely.

Accordingly, given the shortcomings of existing approaches and devices, a need exists for unconventional approaches and devices that more efficiently and more effectively enable users to occlude blood flow.

This Background is provided to introduce a brief context for the Detailed Description that follows. This Background is not intended to be an aid in determining the scope of the claimed subject matter nor be viewed as limiting the claimed subject matter to implementations that solve any or all of the shortcomings or problems presented above.

### SUMMARY

The disclosed embodiments provide unconventional mechanisms for tightening a lace.

Some disclosed embodiments provide unconventional mechanisms, devices, and methods for occlusion of blood flow.

Consistent with the present embodiments, a tightening mechanism comprises a spool being configured to accept a portion of at least one lace. The tightening mechanism comprises a ratchet wheel operably connected to the spool, the ratchet wheel including a plurality of ratchet teeth. The tightening mechanism comprises a knob operably connected to the ratchet wheel, the knob being configured for manually winding a portion of the at least one lace onto the spool in a winding direction. The tightening mechanism comprises a first pawl arm comprising a first pawl, the first pawl being configured to engage with each of the plurality of ratchet teeth to prevent the spool from moving in a direction opposite to the winding direction. The tightening mechanism comprises a second pawl arm comprising a second pawl, the second pawl being configured to engage with each of the plurality of ratchet teeth to prevent the spool from moving in the direction opposite to the winding direction. The tightening mechanism comprises a spring being configured to bias the first pawl and the second pawl towards the ratchet wheel.

The knob may comprise a center portion having a circular shape. The knob may comprise two extensions, each of the two extensions extending radially outward from the center portion.

The center portion and the two extensions may each comprise a plurality of protrusions.

The spring may comprise a torsion spring. The first pawl arm may comprise a first boss being configured to restrain a first end of the torsion spring.

The spring may comprise a torsion spring. The second pawl arm may comprise a second boss being configured to restrain a second end of the torsion spring.

The tightening mechanism may comprise a toggle arm being configured to alternate disengagement of the first pawl and the second pawl from the ratchet wheel.

The toggle arm may comprise a release lever that when actuated in a first release direction, may be configured to release the first pawl from the ratchet wheel, and when actuated in a second release direction, may be configured to release the second pawl from the ratchet wheel.

The release lever may comprise a plurality of protrusions.

The tightening mechanism may comprise a first pawl arm pivot pin, the first pawl arm being configured to pivot about the first pawl arm pivot pin.

The tightening mechanism may comprise a second pawl arm pivot pin, the second pawl arm being configured to pivot about the second pawl arm pivot pin.

The tightening mechanism may comprise a toggle arm pivot pin, the toggle arm being configured to pivot about the toggle arm pivot pin.

The tightening mechanism may comprise a lace anchor pin, the lace anchor pin being configured to anchor the at least one lace to the tightening mechanism.

The tightening mechanism may comprise a lower housing.

The lower housing may be configured to be fastened to an article.

The article may comprise a base of a tourniquet.

The tightening mechanism may comprise an upper housing, the upper housing being configured to connect to the lower housing.

The upper housing may be configured to be fastened to the lower housing.

The upper housing may be connected to the lower housing, the upper housing and the lower housing together may be configured to house the spool, the ratchet wheel, the first pawl arm, the second pawl arm, and the spring.

The tightening mechanism may comprise an upper housing and a lower housing, the upper housing may be connected to the lower housing, the upper housing and the lower housing together may be configured to house the toggle arm.

The upper housing may define a release lever aperture.

The upper housing may define a lace path.

The lace path may include a plurality of lace apertures.

The tightening mechanism may comprise a knob bolt. The knob bolt may be configured to fasten the spool and the ratchet wheel to the knob.

Consistent with the present embodiments, a wearable device comprises a tourniquet. The tourniquet comprises a base. The tourniquet comprises a tightening mechanism connected to the base. The tightening mechanism comprises a spool being configured to accept a first portion of a lace. The tightening mechanism comprises a knob being configured for manually winding the first portion of the lace onto the spool. The tightening mechanism comprises an anchor point being configured to anchor a second portion of the lace. The tourniquet comprises a tunnel strap. The tourniquet comprises a tunnel sled connected to a first end of the tunnel strap. The tunnel sled comprises a first lace guide being configured to guide the lace from the anchor point directly to the spool. The tunnel sled comprises a second lace guide being configured to guide the lace from the anchor point directly to the spool. The wearable device comprises a wearable item quick release connector connected to the tourniquet and being configured to removably connect to a wearable item.

The wearable item may comprise at least one of a ballistic plate carrier, a backpack, a belt, or a harness.

The wearable item quick release connector may be magnetic.

The wearable device may comprise an outer cuff being configured to cover at least a portion of the tourniquet.

The tightening mechanism may comprise a housing being configured to house the spool, the first portion of the lace, and the second portion of the lace.

The housing may be fastened to the base.

The tourniquet may comprise a tunnel strap quick release connector connected to a second portion of the tunnel strap, the tunnel strap quick release connector may be configured to removably connect to a second end of the base.

The tunnel strap quick release connector may comprise a G hook.

The tunnel strap quick release connector may be magnetic.

The base may comprise a base loop being configured to receive a tunnel strap quick release connector.

The base may comprise a first base loop being configured to receive a tunnel strap quick release connector. The base may comprise a second base loop being configured to receive the tunnel strap quick release connector. The first base loop may be configured for larger limbs, and the second base loop may be configured for smaller limbs.

Consistent with the present embodiments, a tourniquet comprises a base. The tourniquet comprises a tightening mechanism connected to the base. The tightening mechanism comprises a spool being configured to accept a first portion of a lace. The tightening mechanism comprises a knob being configured for manually winding the first portion of the lace onto the spool. The tightening mechanism comprises an anchor point being configured to anchor a second portion of the lace. The tourniquet comprises a tunnel strap. The tourniquet comprises a tunnel sled connected to a first end of the tunnel strap. The tunnel sled comprises a first lace guide being configured to guide the lace from the anchor point directly to the spool. The tunnel sled comprises a second lace guide being configured to guide the lace from the anchor point directly to the spool.

The tourniquet may comprise an outer cuff being configured to cover at least a portion of the base.

The tightening mechanism may comprise a release lever.

The tightening mechanism may comprise a housing. The housing may be configured to house the spool, the first portion of the lace, and the second portion of the lace.

The housing may be fastened to the base.

The tourniquet may comprise a tunnel strap quick release connector connected to a second portion of the tunnel strap, the tunnel strap quick release connector may be configured to removably connect to a second end of the base.

The tunnel strap quick release connector may comprise a G hook.

The tunnel strap quick release connector may be magnetic.

The base may comprise a base loop. The base loop may be configured to receive a tunnel strap quick release connector.

The base may comprise a first base loop configured to receive a tunnel strap quick release connector. The base may comprise a second base loop configured to receive the tunnel strap quick release connector. The first base loop may be configured for larger limbs, and the second base loop may be configured for smaller limbs.

The foregoing general description and the following detailed description disclose example embodiments which are explanatory only and are not restrictive of the claims.

### BRIEF DESCRIPTION OF THE SEVERAL VIEWS OF THE DRAWINGS

The accompanying drawings, which are incorporated in and constitute a part of this specification, illustrate several embodiments and, together with the description, serve to explain the disclosed principles. In the drawings:

FIGS. 1A and 1B illustrate various views of an example tightening mechanism consistent with disclosed embodiments.

FIGS. 2A and 2B illustrate various exploded views of an example tightening mechanism consistent with disclosed embodiments.

FIGS. 3A and 3B illustrate example functionality of an example tightening mechanism consistent with disclosed embodiments.

FIG. 4 illustrates an exploded view of an example tightening mechanism configured for quicker release consistent with disclosed embodiments.

FIG. 5A illustrates example lace guides as part of an example tunnel sled connected to an example tightening mechanism consistent with disclosed embodiments.

FIG. 5B illustrates example lace guides as part of an example tunnel sled connected to an example tightening mechanism secured to an example base consistent with disclosed embodiments.

FIG. 6A illustrates example lace guides as part of an example tunnel sled connected to an example tightening mechanism secured to an example base consistent with disclosed embodiments.

FIG. 6B illustrates an example tunnel sled connected to an example tightening mechanism secured to an example base with example base loops consistent with disclosed embodiments.

FIG. 7A illustrates an example tunnel sled connected to an example tightening mechanism secured to an example base with an example tunnel strap quick release connector connected to an example first base loop consistent with disclosed embodiments.

FIG. 7B illustrates an example tightening mechanism secured to an example base with an example tunnel strap quick release connector connected to an example second base loop consistent with disclosed embodiments.

FIG. 8A illustrates a perspective view of an example tourniquet with an example tunnel strap quick release connector connected to an example first base loop consistent with disclosed embodiments.

FIG. 8B illustrates a perspective view of an example tourniquet with an example tunnel strap quick release connector connected to an example first base loop and an outer cuff consistent with disclosed embodiments.

FIG. 9 illustrates a side view of an example tourniquet with an example tunnel strap quick release connector connected to an example second base loop consistent with disclosed embodiments.

FIG. 10 illustrates a perspective view of an example tourniquet package consistent with disclosed embodiments.

FIG. 11 illustrates a low point of view of an example tourniquet package consistent with disclosed embodiments.

FIG. 12 illustrates the tightening of an example tunnel strap of an example tourniquet consistent with disclosed embodiments.

FIG. 13 illustrates the winding of an example knob of an example tightening mechanism of an example tourniquet consistent with disclosed embodiments.

FIG. 14 illustrates the winding of an example knob of an example tightening mechanism of an example tourniquet applied over example clothing consistent with disclosed embodiments.

FIG. 15 illustrates an example tourniquet applied over example clothing consistent with disclosed embodiments.

FIG. 16 illustrates a first view of example wearable devices attached to example wearable items consistent with disclosed embodiments.

FIG. 17 illustrates a second view of example wearable devices attached to example wearable items consistent with disclosed embodiments.

FIG. 18 illustrates a third view of example wearable devices attached to example wearable items consistent with disclosed embodiments.

FIG. 19 illustrates a first view of an example wearable device and example wearable items consistent with disclosed embodiments.

FIG. 20 illustrates a second view of an example wearable device and example wearable items consistent with disclosed embodiments.

FIG. 21 illustrates an example wearable device attached to an example wearable item consistent with disclosed embodiments.

FIG. 22 illustrates a first view of example wearable devices attached to an example backpack with an example deployable front ballistic plate carrier consistent with disclosed embodiments.

FIG. 23 illustrates a second view of an example wearable device attached to an example backpack with an example deployable front ballistic plate carrier consistent with disclosed embodiments.

FIG. 24 illustrates an example backpack with an example deployable front ballistic plate carrier consistent with disclosed embodiments.

FIG. 25 illustrates an example wearable device attached to an example backpack with an example deployable front ballistic plate carrier in a deployed position consistent with disclosed embodiments.

FIG. 26 illustrates an example wearable device attached to an example wearable item consistent with disclosed embodiments.

### DETAILED DESCRIPTION OF EMBODIMENTS

Disclosed embodiments provide unconventional mechanisms for tightening a lace.

Some disclosed embodiments provide a tightening mechanism. The tightening mechanism may comprise a first pawl and a second pawl. The tightening mechanism may be configured to alternate pawl engagement with ratchet teeth. The second pawl may be misclocked by half a tooth from the first pawl such that only one of the first pawl or the second pawl may be engaged with one of the plurality of ratchet teeth at any one time. Therefore, the ratchet teeth may be configured for more strength compared to a tightening mechanism with simultaneous pawl engagement given the same tightening resolution. For example, the ratchet teeth may be constructed with more material to achieve more strength. Similarly, the tightening mechanism may be configured for a finer resolution compared to a tightening mechanism with simultaneous pawl engagement given the same sized ratchet teeth.

Some disclosed embodiments provide unconventional mechanisms, devices, and methods for occlusion of blood flow.

Some disclosed embodiments provide a tourniquet. The tourniquet may comprise at least one lace. A lace may form a loop. The loop may be formed with a splice. The loop may be formed with or without a knot. The tourniquet may be structurally configured to operate upon the event one portion of a lace becomes damaged or cut. The tourniquet may comprise a base. The tourniquet may be foldable. A foldable tourniquet may be more compact and easier to store and/or carry in a folded position. The tourniquet may be configured for rapid deployment. The tourniquet may be configured for ease of use. The tourniquet may be configured for ease of use by a novice. A plurality of tourniquets may be stored in a first response vehicle. A plurality of tourniquets may be stored in locations where mass casualty incidents could occur such as, but not limited to, hospitals, airports, train stations, churches, schools, stadiums, concert venues, and/or other types of locations where mass casualty incidents could occur.

Some disclosed embodiments provide at least one wearable device. A wearable device may be configured for rapid deployment. The wearable device may comprise a tourniquet.

The wearable device may be configured such that the tourniquet may be located over an artery of a wearer for effective occlusion of blood flow if necessary. The wearable device may be configured for ease of use. The wearable device may be configured for ease of use by a novice.

Some disclosed embodiments provide at least one strap. The at least one strap may comprise at least one organic material.

Embodiments consistent with the present disclosure may include a tightening mechanism. The tightening mechanism may comprise a spool. The spool may be configured to accept a portion of at least one lace. The tightening mechanism may comprise a ratchet wheel. The ratchet wheel may be operably connected to the spool. The ratchet wheel may include a plurality of ratchet teeth. The tightening mechanism may comprise a knob. The knob may be operably connected to the ratchet wheel. The knob may be operably connected to the ratchet wheel through employment of a slip clutch. The slip clutch may be configured to engage the knob to the ratchet wheel when the knob is rotated in a winding direction. The slip clutch may be configured to disengage the knob from the ratchet wheel when the knob is rotated in a direction opposite to the winding direction. The knob may be configured for manually winding a portion of the at least one lace onto the spool in a winding direction. The tightening mechanism may comprise a first pawl arm comprising a first pawl. The first pawl may be configured to engage with each of the plurality of ratchet teeth to prevent the spool from moving in a direction opposite to the winding direction. The first pawl may be configured to engage with each of the plurality of ratchet teeth one ratchet tooth at a time. The tightening mechanism may comprise a second pawl arm comprising a second pawl. The second pawl may be configured to engage with each of the plurality of ratchet teeth to prevent the spool from moving in the direction opposite to the winding direction. The second pawl may be configured to engage with each of the plurality of ratchet teeth one ratchet tooth at a time. The second pawl may be misclocked by half a tooth from the first pawl such that only one of the first pawl or the second pawl may be engaged with one of the plurality of ratchet teeth at any one time. The tightening mechanism may comprise a spring. The spring may be configured to bias the first pawl and the second pawl towards the ratchet wheel. The spring may comprise a single spring.

In some embodiments, a knob may comprise a center portion having a circular shape. The knob may comprise two or more extensions. Each of the extensions may extend radially outward from the center portion. The center portion may comprise a plurality of protrusions. Each of the extensions may comprise a plurality of protrusions. The protrusions may be created through employment of a mold. The protrusions may be created by removing material between the protrusions. The protrusions may be configured for tactile purchase. Tactile purchase may be useful when manually operating the knob in a winding direction.

In some embodiments, a knob may comprise one or more directional arrows. The directional arrows may be configured to indicate a winding direction. The directional arrows may be located on extensions of the knob.

In some embodiments, a spring may comprise a torsion spring. A first pawl arm may comprise a first boss. The first boss may be configured to restrain a first end of the torsion spring. The first end may comprise a first reaction end. A second pawl arm may comprise a second boss. The second boss may be configured to restrain a second end of the torsion spring. The second end may comprise a second reaction end.

In some embodiments, a tightening mechanism may comprise a toggle arm. The toggle arm may be configured to alternate disengagement of a first pawl and a second pawl from a ratchet wheel.

In some embodiments, a first pawl arm may comprise a first cam. A second pawl arm may comprise a second cam. A toggle arm may be configured to disengage the first pawl when pressure is applied to the first cam. The toggle arm may be configured to disengage the second pawl when pressure is applied to the second cam.

In some embodiments, when a ratchet wheel of a tightening mechanism may be moving in a tightening direction, a toggle arm may be configured to follow a first cam and a second cam as a first pawl and a second pawl alternate engagement with ratchet teeth.

In some embodiments, a toggle arm may comprise a release lever. The release lever may be configured for actuation in a first release direction. The release lever may be configured for actuation in a second release direction. The first release direction may oppose the second release direction. When actuated in the first release direction, the release lever may be configured to release a first pawl from a ratchet wheel. When actuated in the second release direction, the release lever may be configured to release a second pawl from the ratchet wheel. The release lever may comprise a plurality of protrusions.

In some embodiments, when a spool may be under tension from at least one lace and a first pawl may be engaged with a ratchet wheel, actuation of a release lever in a first release direction may cause the spool to rotate in a direction opposite to a winding direction. The spool may rotate in the direction opposite to the winding direction until stopped by a second pawl. When the spool may be under tension from at least one lace and the second pawl may be engaged with the ratchet wheel, actuation of the release lever in a second release direction may cause the spool to rotate in the direction opposite to the winding direction. The spool may rotate in the direction opposite to the winding direction until stopped by the first pawl.

In some embodiments, when a release lever may be actuated unintentionally in one of a first release direction or a second release direction, a tightening mechanism may be configured to maintain ability for manual winding of a portion of at least one lace onto a spool in a winding direction through employment of a knob. In this scenario, only one of a first pawl or a second pawl may be configured to engage with each of the plurality of ratchet teeth to prevent the spool from moving in a direction opposite to the winding direction when the knob may be moved in the winding direction.

In some embodiments, a tightening mechanism may comprise a first pawl arm pivot pin. A first pawl arm may be configured to pivot about the first pawl arm pivot pin.

In some embodiments, a tightening mechanism may comprise a second pawl arm pivot pin. A second pawl arm may be configured to pivot about the second pawl arm pivot pin.

In some embodiments, a tightening mechanism may comprise a toggle arm pivot pin. A toggle arm may be configured to pivot about the toggle arm pivot pin.

In some embodiments, a lower housing may be configured to be fastened to an article. One side of the lower housing may be configured to be fastened to an article. The article may comprise a base of a tourniquet.

In some embodiments, a tightening mechanism may comprise an upper housing. The upper housing may be configured to connect to a lower housing. The upper housing may be configured to be fastened to a lower housing. The upper housing may define a lace path. The lace path may include one or more lace apertures. For example, the lace path may include one aperture configured to receive at least one lace. In another example, the lace path may include three apertures. In this example, one anchor aperture may be configured to receive at least one lace from an anchor point. Each of two spool apertures may be configured to receive at least one lace from a spool. In yet another example, the lace path may include four apertures. In this example, each of two anchor apertures may be configured to receive at least one lace from an anchor point. Each of two spool apertures may be configured to receive at least one lace from a spool.

In some embodiments, an upper housing may be connected to a lower housing. The upper housing and the lower housing together may be configured to house a spool. The upper housing and the lower housing together may be configured to house a ratchet wheel. The upper housing and the lower housing together may be configured to house a first pawl arm. The upper housing and the lower housing together may be configured to house a second pawl arm. The upper housing and the lower housing together may be configured to house a spring. The upper housing and the lower housing together may be configured to house a toggle arm.

In some embodiments, an upper housing may define a release lever aperture. The release lever aperture may be configured to receive a release lever.

In some embodiments, an upper housing may define a loop slot. The loop slot may be configured to engage with a loop of material attached to a base of an article. The article may comprise a tourniquet. The loop slot may be configured to removably engage with the loop of material. A base loop may comprise the loop of material. A first base loop of a plurality of base loops may comprise the loop of material.

In some embodiments, a tightening mechanism may comprise a bolt. The bolt may be configured to fasten a spool and a ratchet wheel to a knob.

Embodiments consistent with the present disclosure may include a tourniquet. The tourniquet may comprise a base. The tourniquet may comprise a tightening mechanism. The tightening mechanism may be connected to the base. The tightening mechanism may comprise a spool. The spool may be configured to accept a first portion of a lace. The tightening mechanism may comprise a knob. The knob may be configured for manually winding the first portion of the lace onto the spool. The tightening mechanism may comprise an anchor point. The anchor point may be configured to anchor a second portion of the lace. The tourniquet may comprise a tunnel strap. The tourniquet may comprise a first lace guide. The first lace guide may be connected to a first end of the tunnel strap. The first lace guide may be configured to guide the lace from the anchor point directly to the spool. The first lace guide may be configured to guide the lace from the anchor point directly to a first side of the spool. The tourniquet may comprise a second lace guide. The second lace guide may be connected to the first end of the tunnel strap. The second lace guide may be configured to guide the lace from the anchor point directly to the spool. The second lace guide may be configured to guide the lace from the anchor point directly to a second side of the spool. The second side of the spool may be opposite to the first side of the spool.

In some embodiments, a tourniquet may comprise an outer cuff. The outer cuff may be configured to cover at least a portion of a first end of a base. The outer cuff may be attached to the base. The outer cuff may be configured to cover at least a portion of a first end of a tunnel strap. The outer cuff may be configured to cover at least a portion of two lace guides. The outer cuff may be structurally configured to cover at least a portion of a lace.

In some embodiments, an outer cuff may be interchangeable. A plurality of distinct outer cuffs may comprise a plurality of distinct patterns. For example, distinct patterns may comprise a specific camouflage pattern and/or a specific set of camouflage colors. The outer cuff may comprise at least one organic material. The outer cuff may comprise printed instructions for use of a wearable device and/or a tourniquet. The outer cuff may comprise one or more fasteners. The one or more fasteners may comprise a snap, a zipper, a hook and loop fastener, and/or any other type of fastener. The outer cuff may comprise an outer hook and loop fastener. The outer hook and loop fastener may be sewn onto the outside of the outer cuff.

In some embodiments, a base may be flexible. The base may comprise nylon webbing. The base may comprise polypropylene webbing. The base may comprise at least one recycled material. The base may be structurally configured to conform to an arm and/or a leg of a wearer. The base may be connected to a tightening mechanism. A second end of the base may be configured to connect to a second portion of a tunnel strap. The second end of the base may form a base loop. Alternatively, a base loop may comprise a loop of material attached to the base. The base loop may be disposed to the second end of the base. The base loop may extend past the second end of the base. The base may comprise a tensile strength of at least 3000 pounds.

In some embodiments, a first base loop may comprise a loop of material attached to a base. The first base loop may be configured to receive a tunnel strap quick release connector. A second base loop may comprise a loop of material attached to the base. The second base loop may be configured to receive the tunnel strap quick release connector. The first base loop may be configured for standard and/or larger limbs. The second base loop may be configured for smaller limbs. The first base loop may have a longer reach than the second base loop. The reach may be measured from the end of a base loop to a point on the base where the base may be connected to a tightening mechanism. When the tunnel strap quick release connector may be connected to the first base loop, the first base loop may be configured to overlap the second base loop. When the tunnel strap quick release connector may be connected to the second base loop, a tourniquet may be configured to occlude blood flow in a limb with a minimum diameter of 15/16″. When the tunnel strap quick release connector may be connected to the first base loop, a tourniquet may be configured to occlude blood flow in a limb with a maximum diameter of 14″.

In some embodiments, a lace may be employed to connect a tightening mechanism to a first end of a tunnel strap. The lace may, for example, comprise ultra-high molecular weight polyethylene fibers. The lace may comprise a hollow core. The lace may, for example, comprise at least one organic material. The lace may be secured to at least one anchor point in a tightening mechanism. The lace may be secured to a spool in the tightening mechanism. The lace may be in the form of a loop connecting at least one anchor point, the first end of the tunnel strap, and the spool. The two tag ends of the lace may be spliced together and/or tied together.

In some embodiments, a first end of a tunnel strap may be connected to two lace guides. The two lace guides may be configured to guide a lace. The two lace guides may comprise at least one organic material. The first end of the tunnel strap and the two lace guides may be structurally configured to slide along the surface of a base as a tightening mechanism may be tightened.

In some embodiments, a second end of a tunnel strap may be structurally configured to connect to a second end of a base. The second end of the tunnel strap may be structurally configured to connect to the second end of the base through employment of a tunnel strap quick release connector. The tunnel strap may be adjustable in length through employment of the tunnel strap quick release connector.

In some embodiments, a base may be structurally configured to receive a second end of a tunnel strap. The second end of the tunnel strap may be received through employment of a tunnel strap loop connector. The tunnel strap loop connector may be connected to the base. The tunnel strap loop connector may be integrated into the base. The tunnel strap may be adjustable in length through employment of the tunnel strap loop connector.

In some embodiments, a tunnel strap quick release connector may be connected to a second portion of a tunnel strap. The second portion may be a portion in between a first end of the tunnel strap and a second end of the tunnel strap. The tunnel strap quick release connector may be configured to removably connect the second portion of the tunnel strap to a second end of a base. The tunnel strap quick release connector may be configured to enable a wearer to adjust the length of the tunnel strap with one hand. The tunnel strap may be adjusted to allow for donning a tourniquet over a variety of extremity sizes. The tunnel strap may be adjusted to allow for donning the tourniquet over clothing, if necessary. The tunnel strap quick release connector may comprise a G hook. The G hook may comprise titanium. The tunnel strap quick release connector may be magnetic. An example of a magnetic quick release connector is a V-BUCKLE by Fidlock.

In some embodiments, a tag end of a second end of a tunnel strap may form a tunnel strap loop. The tunnel strap loop may prevent the tag end of the tunnel strap from being removed from a tunnel strap quick release connector. The tunnel strap loop may be employed by a wearer to pull the tunnel strap tight with less grip strength than by grasping the tunnel strap without a tunnel strap loop. The tunnel strap loop may be employed by a wearer to pull the tunnel strap tight without having to rely on grip strength. The tunnel strap loop may comprise one or more brightly colored materials. The tunnel strap loop may comprise one or more instructions. The one or more instructions may be printed one of the brightly colored materials. For example, the instructions may comprise “pull tight”. The instructions may comprise an arrow configured to indicate a pulling direction. The tunnel strap loop may comprise a designated location for noting time of application of a tourniquet. The tunnel strap loop may comprise a designated location for noting a blood type of a wearer.

Some embodiments may provide a tunnel strap extender. A first end of the tunnel strap extender may comprise an extender loop. The extender loop may be configured to receive a tunnel strap quick release connector. A second end of the tunnel strap extender may comprise an extender quick release connector. The extender quick release connector may be configured to removably connect the second end of the tunnel strap extender to a second end of a base. The extender quick release connector may be configured to enable a wearer to adjust the length of the tunnel strap extender with one hand. The tunnel strap extender may be adjusted to allow for donning a tourniquet over a variety of extremity sizes. The tunnel strap extender may be adjusted to allow for donning the tourniquet over clothing, if necessary. The tunnel strap extender may be added to a tourniquet to allow for employment of the tourniquet to stabilize a hip of a wearer. The tunnel strap extender may be added to a tourniquet to allow for employment of the tourniquet as a junctional tourniquet. The tunnel strap extender may be added to a tourniquet to allow for employment of the tourniquet as an abdominal tourniquet. The extender quick release connector may comprise a G hook.

In some embodiments, a second end of a tunnel strap may comprise a hook and loop fastener. The hook and loop fastener may be sewn onto the tunnel strap. The hook and loop fastener may be configured to connect to an outer cuff. For example, when the application of a tourniquet has occluded blood flow in a limb of a wearer, the second end of the tunnel strap may be wrapped around the limb one or more times and secured to an outer hook and loop fastener of the outer cuff. This may prevent the second end from snagging on items in the surrounding environment.

Some embodiments may provide a hook and loop fastener. The hook and loop fastener may comprise alternating rows of hook material and loop material. This alternating hook and loop configuration may provide a greater range of adjustability over a standard configuration comprising one section of hook material and one section of loop material.

In some embodiments, a base may comprise at least one anti-slip material. The at least one anti-slip material may be on a side of the base that is opposite the side attached to a tightening mechanism. The at least one anti-slip material may be configured to provide additional traction against an extremity and/or clothing of a wearer. The at least one anti-slip material may comprise slip not grip fabric. The at least one anti-slip material may comprise a polyester fabric. The at least one anti-slip material may comprise a plurality of protrusions. The plurality of protrusions may comprise Polyvinyl Chloride (PVC).

In some embodiments, a tightening mechanism may comprise a housing. The housing may be configured to house a spool. The housing may be configured to house a first portion of a lace. The housing may be configured to house an anchor point. The housing may be configured to house a second portion of a lace. The housing may be fastened to a base.

In some embodiments, a tourniquet may be structurally configured to enable a user to apply a mechanical advantage to apply sufficient pressure to occlude blood flow. The tourniquet may be structurally configured to apply circumferential pressure to occlude blood flow.

In some embodiments, a tourniquet may be configured to weigh less than 4 ounces.

In some embodiments, a tourniquet may be configured to conform to one or more Committee on Tactical Combat Casualty Care (CoTCCC) guidelines. For example, the tourniquet may be configured to fit within a package size of 1.75″×2.25″×6.5″.

Embodiments consistent with the present disclosure may include a wearable device. The wearable device may comprise a tourniquet. The tourniquet may comprise a base. The tourniquet may comprise a tightening mechanism. The tightening mechanism may be connected to the base. The tightening mechanism may comprise a spool. The spool may be configured to accept a first portion of a lace. The tightening mechanism may comprise a knob. The knob may be configured for manually winding the first portion of the lace onto the spool. The tightening mechanism may comprise an anchor point. The anchor point may be configured to anchor a second portion of the lace. The tourniquet may comprise a tunnel strap. The tourniquet may comprise a first lace guide. The first lace guide may be connected to a first end of the tunnel strap. The first lace guide may be configured to guide the lace from the anchor point directly to the spool. The first lace guide may be configured to guide the lace from the anchor point directly to a first side of the spool. The tourniquet may comprise a second lace guide. The second lace guide may be connected to the first end of the tunnel strap. The second lace guide may be configured to guide the lace from the anchor point directly to the spool. The second lace guide may be configured to guide the lace from the anchor point directly to a second side of the spool. The second side of the spool may be opposite to the first side of the spool. The wearable device may comprise a wearable item quick release connector. The wearable item quick release connector may be connected to the tourniquet. The wearable item quick release connector may be configured to removably connect to a wearable item.

In some embodiments, a wearable item may comprise a ballistic plate carrier. The ballistic plate carrier may comprise two shoulder straps. Each of the two shoulder straps may be configured to connect to a wearable item quick release connector.

In some embodiments, a wearable item may comprise a backpack. The backpack may comprise two shoulder straps. Each of the two shoulder straps may be configured to connect to a wearable item quick release connector. The backpack may comprise a rear ballistic plate carrier. The backpack may be structurally configured to connect to a deployable front ballistic plate carrier. The deployable front ballistic plate carrier may be deployed on the front of a wearer without impacting one or more connected tourniquets.

In some embodiments, a wearable item may comprise a belt. The belt may be configured to connect to one or more wearable item quick release connectors.

In some embodiments, a wearable item may comprise a harness. The harness may be configured to connect to one or more wearable item quick release connectors. The harness may comprise two shoulder straps. Each of the two shoulder straps may be configured to connect to a wearable item quick release connector.

In some embodiments, a wearable device may comprise a wearable item strap. The wearable item strap may be connected to a base of a tourniquet. The wearable item strap may be connected to the base through employment of a single pivot. The single pivot may, for example, comprise a single rivet or a single fastener. The wearable item strap may comprise a wearable item quick release connector. Alternatively, the wearable item quick release connector may be connected directly to the base. The wearable item quick release connector may be connected directly to the base through employment of a single pivot.

Some embodiments provide a wearable item quick release connector. The wearable item quick release connector may be configured to removably connect to a wearable item. The wearable item quick release connector may be magnetic. The wearable item quick release connector may be structurally configured to be adjustable to properly place a tourniquet on an extremity of a wearer. The wearable item quick release connector may enable the tourniquet to remain in place while the wearable item may be adjusted or removed from the wearer.

In some embodiments, wearable devices may be connected to wearable items such that tourniquets are automatically located at proximal points of extremities. This automatic location may eliminate the need to don the tourniquet after an injury is sustained.

In some embodiments, a wearable device may comprise an outer cuff. The outer cuff may be structurally configured to cover at least a portion of a tourniquet. The portion of the tourniquet may comprise at least a portion of a first end of a base. The portion of the tourniquet may comprise at least a portion of at least one lace. The portion of the tourniquet may comprise at least a portion of two lace guides. The portion of the tourniquet may comprise at least a portion of a first end of a tunnel strap.

Reference will now be made in detail to example embodiments, examples of which are illustrated in the accompanying drawings and disclosed herein. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

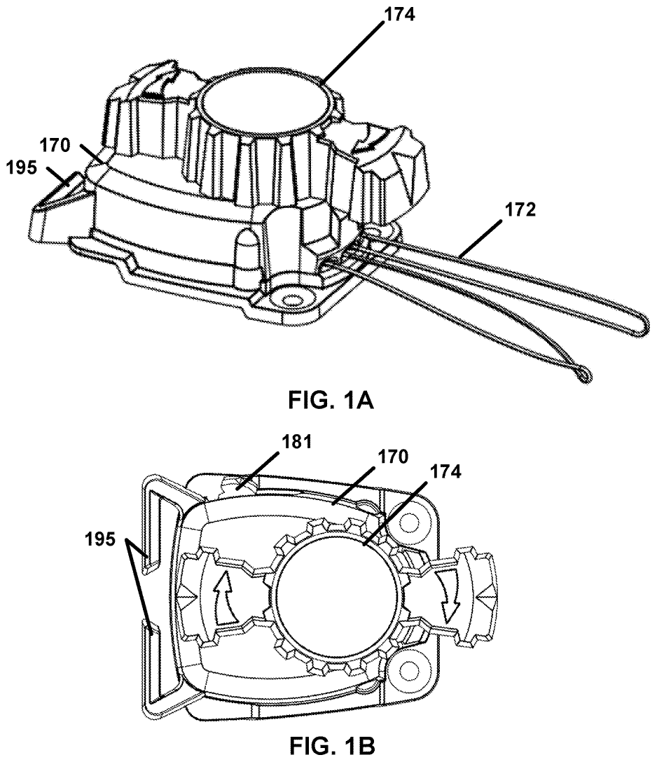

FIGS. 1A and 1B illustrate various views of an example tightening mechanism consistent with disclosed embodiments. A tightening mechanism may comprise a housing 170. A housing 170 may comprise an upper housing and a lower housing. A tightening mechanism may comprise a knob 174. A tightening mechanism may comprise a base loop retainer 195. A base loop retainer 195 may be configured to retain a base loop. A tightening mechanism may comprise at least one lace 172. A tightening mechanism may comprise a release lever 181.

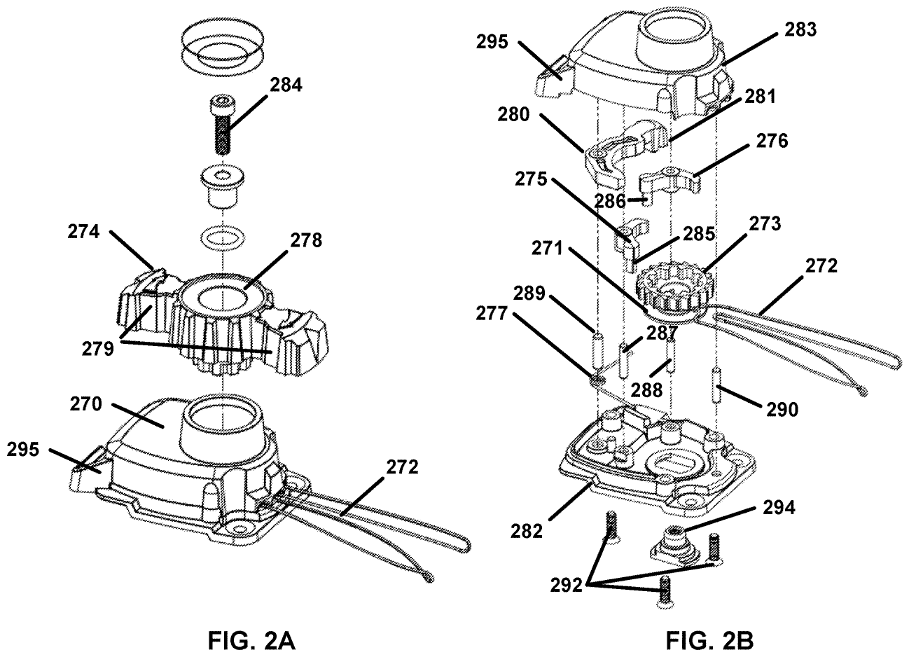

FIGS. 2A and 2B illustrate various exploded views of an example tightening mechanism consistent with disclosed embodiments. A tightening mechanism may comprise a housing 270. A housing 270 may comprise a lower housing 282 and an upper housing 283. A tightening mechanism may comprise a base loop retainer 295. A tightening mechanism may comprise a knob 274. A knob 274 may comprise a center portion 278. A knob 274 may comprise a plurality of extensions 279. A tightening mechanism may comprise a knob bolt 284. A tightening mechanism may comprise a spool 271. A tightening mechanism may comprise at least one lace 272. A tightening mechanism may comprise a ratchet wheel 273. A tightening mechanism may comprise a first pawl arm 275. A first pawl arm 275 may comprise a first boss 285. A tightening mechanism may comprise a second pawl arm 276. A second pawl arm 276 may comprise a second boss 286. A tightening mechanism may comprise a spring 277. A tightening mechanism may comprise a toggle arm 280. A toggle arm 280 may comprise a release lever 281. A tightening mechanism may comprise a first pawl arm pivot pin 287. A tightening mechanism may comprise a second pawl arm pivot pin 288. A tightening mechanism may comprise a toggle arm pivot pin 289. A tightening mechanism may comprise a lace anchor pin 290. A tightening mechanism may comprise one or more housing screws 292. One or more housing screws 292 may be employed to connect a lower housing 282 to a an upper housing 283. A tightening mechanism may comprise a knob bolt nut 294. A knob bolt nut 294 may be configured to receive a knob bolt 284.

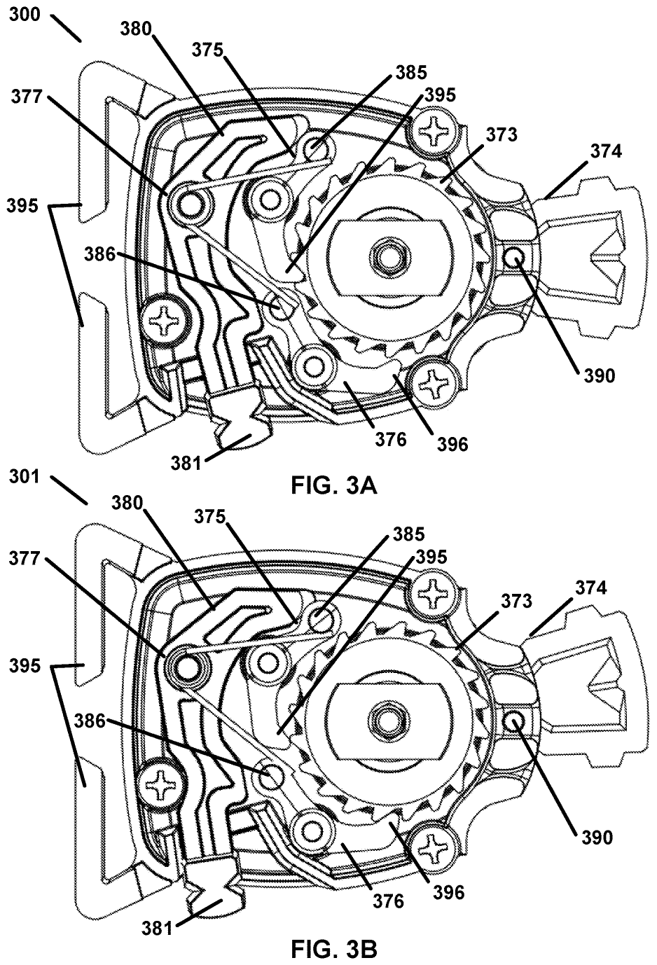

FIGS. 3A and 3B illustrate example functionality of an example tightening mechanism consistent with disclosed embodiments. In FIG. 3A, the tightening mechanism in a first toggle arm position 300 illustrates a first pawl 395 of a first pawl arm 375 engaged between two ratchet teeth of a ratchet wheel 373. A second pawl 396 of a second pawl arm 376 may be disengaged with the ratchet teeth. In this position, only the first pawl 395 may prevent the ratchet wheel 373 from moving in a direction opposite to a winding direction. In FIG. 3B, the tightening mechanism in a second toggle arm position 301 illustrates the second pawl 396 of the second pawl arm 376 engaged between two ratchet teeth. The first pawl 395 of the first pawl arm 375 may be disengaged with the ratchet teeth. In this position, only the second pawl 396 may prevent the ratchet wheel 373 from moving in a direction opposite to a winding direction. The tightening mechanism may comprise a knob 374. The tightening mechanism may comprise a spring 377. The tightening mechanism may comprise a toggle arm 380. The toggle arm 380 may comprise a release lever 381. The first pawl arm 375 may comprise a first boss 385. The second pawl arm 376 may comprise a second boss 386. The tightening mechanism may comprise a lace anchor pin 390. The tightening mechanism may comprise a base loop retainer 395.

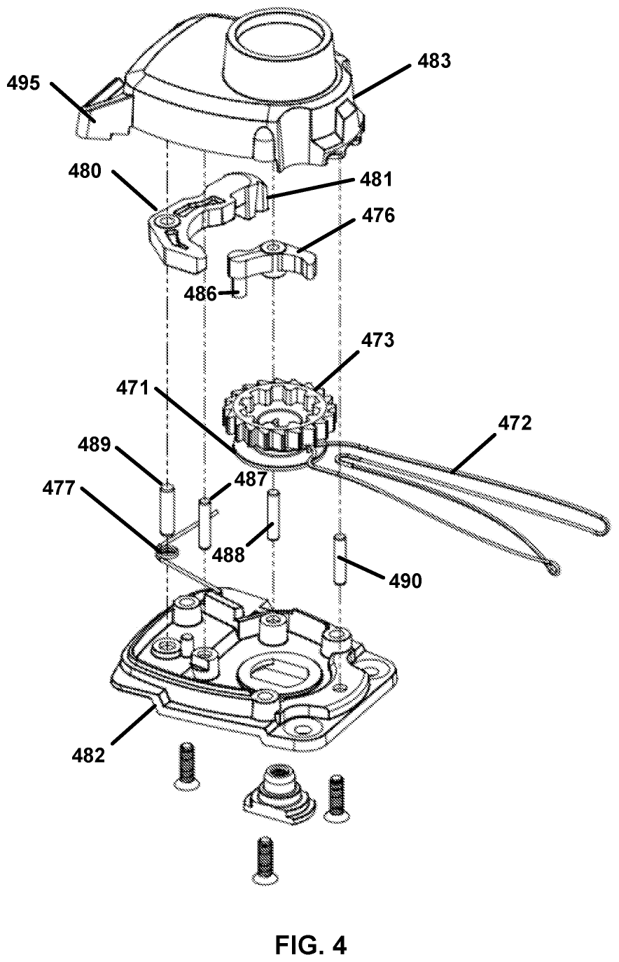

FIG. 4 illustrates an exploded view of an example tightening mechanism configured for quicker release consistent with disclosed embodiments. For example, a tightening mechanism configured for a quicker release may be employed for non-emergency use. For example, a tightening mechanism configured for a quicker release may be employed for training purposes. A tightening mechanism configured for quicker release may comprise a housing 470. The housing 470 may comprise a lower housing 482 and an upper housing 483. A tightening mechanism configured for quicker release may comprise a base loop retainer 495. A tightening mechanism configured for quicker release may comprise a knob 474. A tightening mechanism configured for quicker release may comprise a spool 471. A tightening mechanism configured for quicker release may comprise at least one lace 472. A tightening mechanism configured for quicker release may comprise a ratchet wheel 473. A tightening mechanism configured for quicker release may comprise a second pawl arm 476. The second pawl arm 476 may comprise a second boss 486. A tightening mechanism configured for quicker release may comprise a spring 477. A tightening mechanism configured for quicker release may comprise a toggle arm 480. The toggle arm 480 may comprise a release lever 481. A tightening mechanism configured for quicker release may comprise a first pawl arm pivot pin 487. Since a first pawl arm is not installed, the first pawl arm pivot pin 487 may be employed to restrain a first end of the spring 477. A tightening mechanism configured for quicker release may comprise a second pawl arm pivot pin 488. A tightening mechanism configured for quicker release may comprise a toggle arm pivot pin 489. A tightening mechanism configured for quicker release may comprise a lace anchor pin 490. When the toggle arm 480 of a tightening mechanism configured for quicker release is in a first toggle arm position (as shown in FIG. 3A), no pawl will prevent the ratchet wheel 473 from moving in a direction opposite to a winding direction. When the toggle arm 480 of a tightening mechanism configured for quicker release is in a second toggle arm position (as shown in FIG. 3B), a second pawl of the second pawl arm 476 is engaged between two ratchet teeth of ratchet wheel 473. In this position, the second pawl prevents the ratchet wheel 473 from moving in a direction opposite to a winding direction.

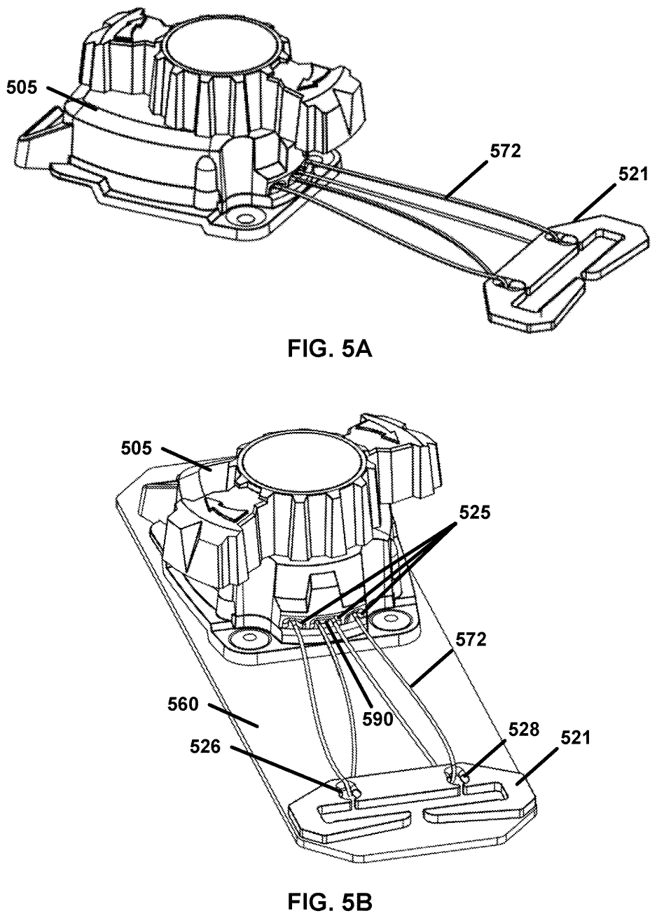

FIG. 5A illustrates example lace guides as part of an example tunnel sled 521 connected to an example tightening mechanism 505 consistent with disclosed embodiments. The tightening mechanism 505 may comprise a lace 572. The lace 572 may form a loop. The tunnel sled 521 may be connected to the tightening mechanism 505 through employment of the lace 572.

FIG. 5B illustrates example lace guides (526 and 528) as part of an example tunnel sled 521 connected to an example tightening mechanism 505 secured to an example base 560 consistent with disclosed embodiments. The tightening mechanism 505 may comprise a lace 572. The lace 572 may form a loop. The tightening mechanism 505 may comprise a housing that defines a lace path. The lace path may include a plurality of lace apertures 525. The tunnel sled 521 may be connected to the tightening mechanism 505 through employment of the lace 572. The Tightening mechanism 505 may comprise an anchor point 590. A first lace guide 526 may be configured to guide a third portion of the lace 572 from the anchor point 590 directly to a spool housed by a housing of the tightening mechanism 505. A second lace guide 528 may be configured to guide a fourth portion of the lace 572 from the anchor point 590 directly to the spool. The tightening mechanism 505 may be fastened to the base 560 as shown.

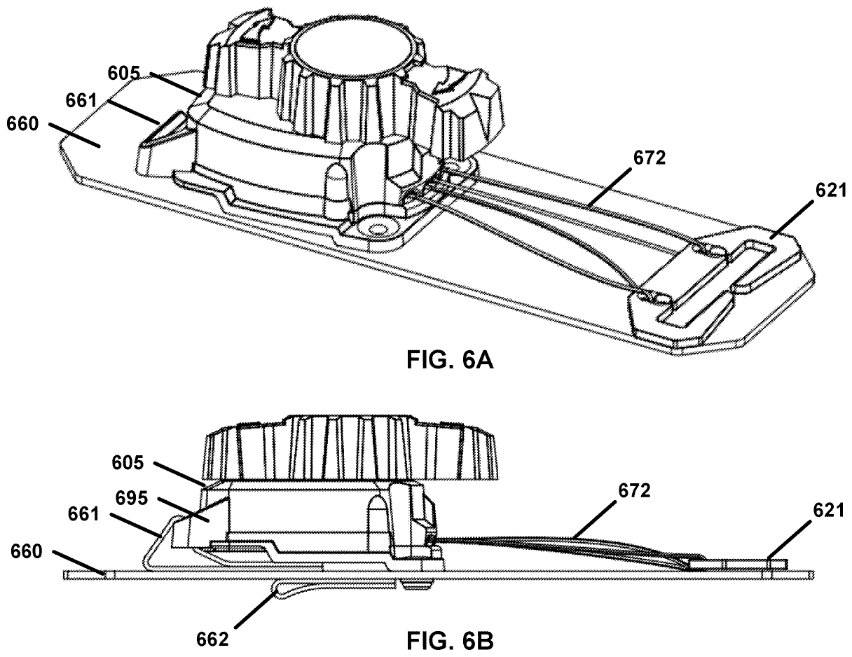

FIG. 6A illustrates example lace guides as part of an example tunnel sled 621 connected to an example tightening mechanism 605 secured to an example base 660 consistent with disclosed embodiments. The tightening mechanism 605 may comprise a lace 672. The tunnel sled 621 may be connected to the tightening mechanism 605 through employment of the lace 672. The tightening mechanism 605 may be fastened to the base 660 as shown. The base 660 may comprise a first base loop 661.

FIG. 6B illustrates an example tunnel sled 621 connected to an example tightening mechanism 605 secured to an example base 660 with example base loops (661 and 662) consistent with disclosed embodiments. The tightening mechanism 605 may comprise a lace 672. The tunnel sled 621 may be connected to the tightening mechanism 605 through employment of the lace 672. The tightening mechanism 605 may be fastened to the base 660 as shown. The base 660 may comprise a first base loop 661. The tightening mechanism 605 may comprise a base loop retainer 695. The base 660 may comprise a second base loop 662.

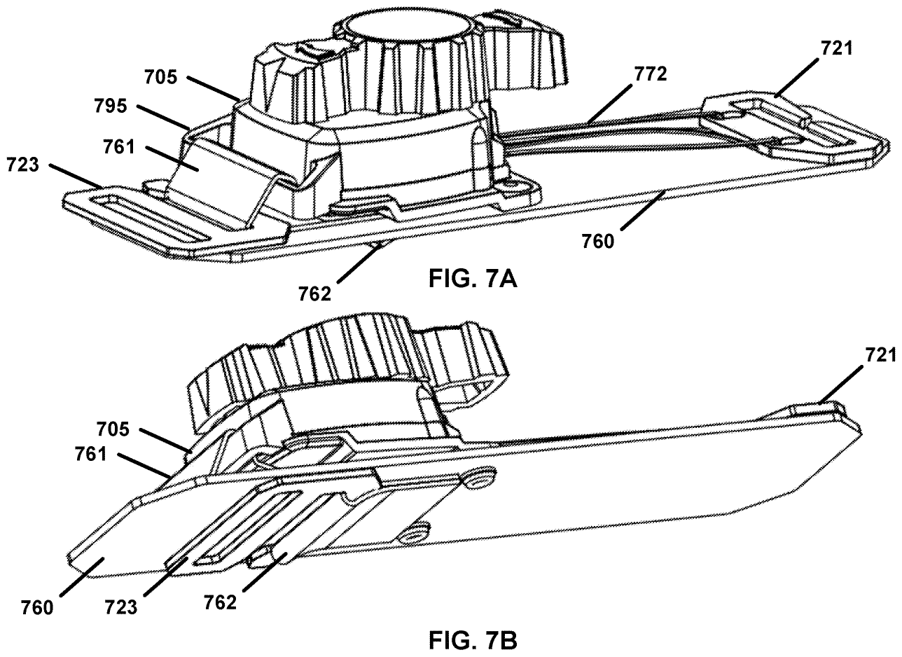

FIG. 7A illustrates an example tunnel sled 721 connected to an example tightening mechanism 705 secured to an example base 760 with an example tunnel strap quick release connector 723 connected to an example first base loop 761 consistent with disclosed embodiments. The tightening mechanism 705 may comprise a lace 772. The tunnel sled 721 may be connected to the tightening mechanism 705 through employment of the lace 772. The tightening mechanism 705 may be fastened to the base 760 as shown. The base 760 may comprise a first base loop 761. The tightening mechanism 705 may comprise a base loop retainer 795. The base 760 may comprise a second base loop 762. The tunnel strap quick release connector 723 may be removably connected to the first base loop 761 as shown.

FIG. 7B illustrates an example tightening mechanism 705 secured to an example base 760 with an example tunnel strap quick release connector 723 connected to an example second base loop 762 consistent with disclosed embodiments. The tightening mechanism 705 may be fastened to the base 760 as shown. The base 760 may comprise a first base loop 761. The base 760 may comprise a second base loop 762. The tunnel strap quick release connector 723 may be removably connected to the second base loop 762 as shown.

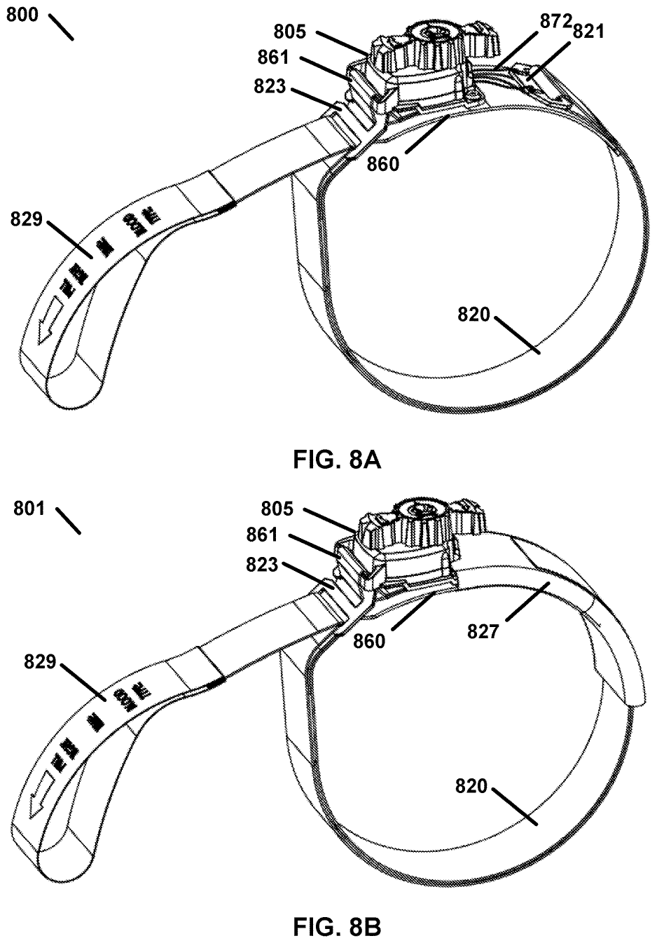

FIG. 8A illustrates a perspective view of an example tourniquet 800 with an example tunnel strap quick release connector 823 connected to an example first base loop 861 consistent with disclosed embodiments. The tourniquet 800 may comprise a tightening mechanism 805. The tightening mechanism 805 may be fastened to a base 860. The tourniquet 800 may comprise a tunnel strap 820. A first end of the tunnel strap 820 may be connected to the tightening mechanism 805 through employment of a tunnel sled 821 and at least one lace 872. A second portion of the tunnel strap 820 may be connected to a tunnel strap quick release connector 823. The length of the tunnel strap 820 may be adjustable. The length of the tunnel strap 820 may be adjusted through employment of a hook and loop fastener. The tunnel strap quick release connector 823 may be removably connected to the first base loop 861. A second end of the tunnel strap 820 may form a tunnel strap loop 829.

FIG. 8B illustrates a perspective view of an example tourniquet 801 with an example tunnel strap quick release connector 823 connected to an example first base loop 861 and an outer cuff 827 consistent with disclosed embodiments. The tourniquet 801 may comprise a tightening mechanism 805. The tightening mechanism 805 may be fastened to a base 860. The tourniquet 800 may comprise a tunnel strap 820. A first end of the tunnel strap 820 may be connected to the tightening mechanism 805 through employment of a tunnel sled and at least one lace. The tourniquet 801 may comprise the outer cuff 827. The outer cuff 827 may be configured to cover the tunnel sled and a portion of the at least one lace. The outer cuff 827 may be configured to cover a portion of the tunnel strap 820. The tunnel sled may be configured to slide along the base 860. A second portion of the tunnel strap 820 may be connected to a tunnel strap quick release connector 823. The length of the tunnel strap 820 may be adjustable. The length of the tunnel strap 820 may be adjusted through employment of a hook and loop fastener. The tunnel strap quick release connector 823 may be removably connected to the first base loop 861. A second end of the tunnel strap 820 may form a tunnel strap loop 829.

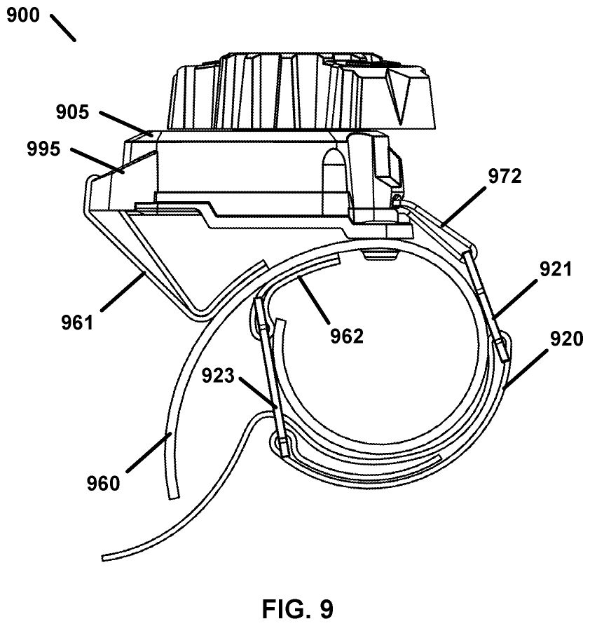

FIG. 9 illustrates a side view of an example tourniquet 900 with an example tunnel strap quick release connector 923 connected to an example second base loop 962 consistent with disclosed embodiments. The tourniquet 900 may comprise a tightening mechanism 905. The tightening mechanism 905 may be fastened to a base 960. The tourniquet 900 may comprise a tunnel strap 920. A first end of the tunnel strap 920 may be connected to the tightening mechanism 905 through employment of a tunnel sled 921 and at least one lace 972. A second portion of the tunnel strap 920 may be connected to a tunnel strap quick release connector 923. The length of the tunnel strap 920 may be adjustable. The length of the tunnel strap 920 may be adjusted by sliding the second portion of the tunnel strap 920 through a slot in the tunnel strap quick release connector 923. The base 960 may comprise a first base loop 961 and the second base loop 962. Tourniquet 900 is shown in a small limb configuration. In the small limb configuration, the tunnel strap quick release connector 923 may be removably connected to the second base loop 962 (as shown). A base loop retainer 995 may be engaged with the first base loop 961. This may limit separation of the tightening mechanism 905 from the base 960 during use of the tourniquet 900.

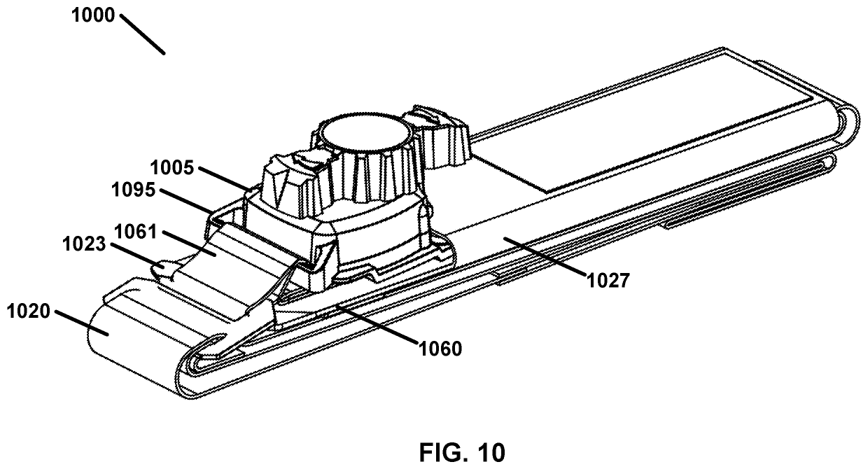

FIG. 10 illustrates a perspective view of an example tourniquet package 1000 consistent with disclosed embodiments. The tourniquet package 1000 may comprise a tourniquet. The tourniquet may comprise a tightening mechanism 1005. The tightening mechanism 1005 may be fastened to a base 1060. The base 1060 may comprise a first base loop 1061. The tourniquet may comprise a tunnel strap 1020. A second portion of the tunnel strap 1020 may be connected to a tunnel strap quick release connector 1023. The tunnel strap quick release connector 1023 may be removably connected to the first base loop 1061. The tourniquet may comprise an outer cuff 1027. The tightening mechanism 1005 may comprise a base loop retainer 1095. The base loop retainer 1095 may be connected to the first base loop 1061. The tourniquet may be folded.

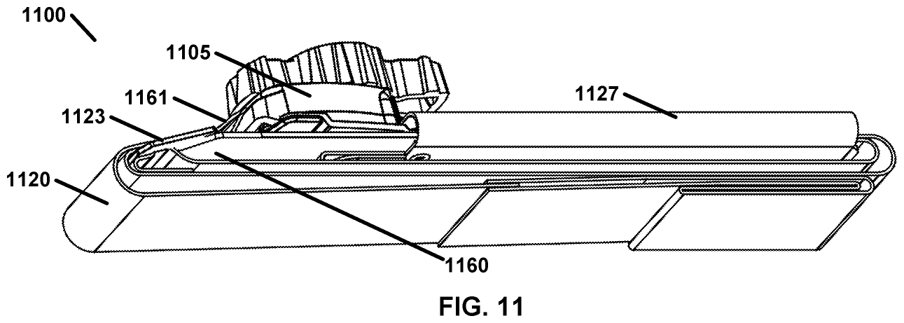

FIG. 11 illustrates a low point of view of an example tourniquet package 1100 consistent with disclosed embodiments. The tourniquet package 1100 may comprise a tourniquet. The tourniquet may comprise a tightening mechanism 1105. The tightening mechanism 1105 may be fastened to a base 1160. The base 1160 may comprise a first base loop 1161. The tourniquet may comprise a tunnel strap 1120. A second portion of the tunnel strap 1120 may be connected to a tunnel strap quick release connector 1123. The tunnel strap quick release connector 1123 may be removably connected to the first base loop 1161. The tourniquet may comprise an outer cuff 1127. The tourniquet may be folded.

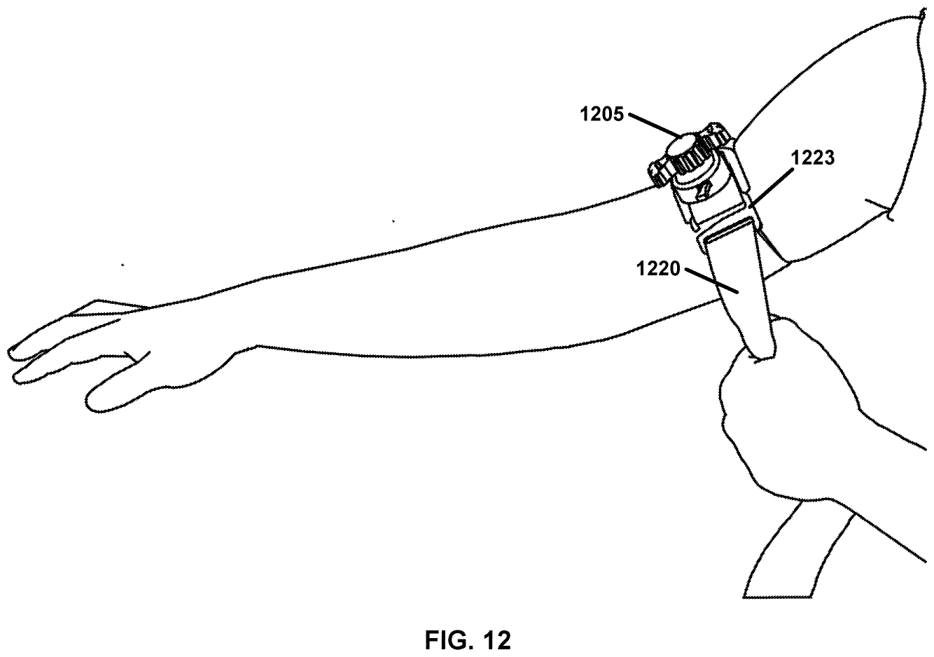

FIG. 12 illustrates the tightening of an example tunnel strap 1220 of an example tourniquet consistent with disclosed embodiments. The tourniquet may comprise a tightening mechanism 1205. The tourniquet may comprise the tunnel strap 1220. A wearer may grasp a second portion of the tunnel strap 1220 to tighten the tunnel strap around an extremity. The second portion of the tunnel strap 1220 may be configured to slide through a tunnel strap quick release connector 1223 during tightening. The tunnel strap quick release connector 1223 may be connected to a base loop.

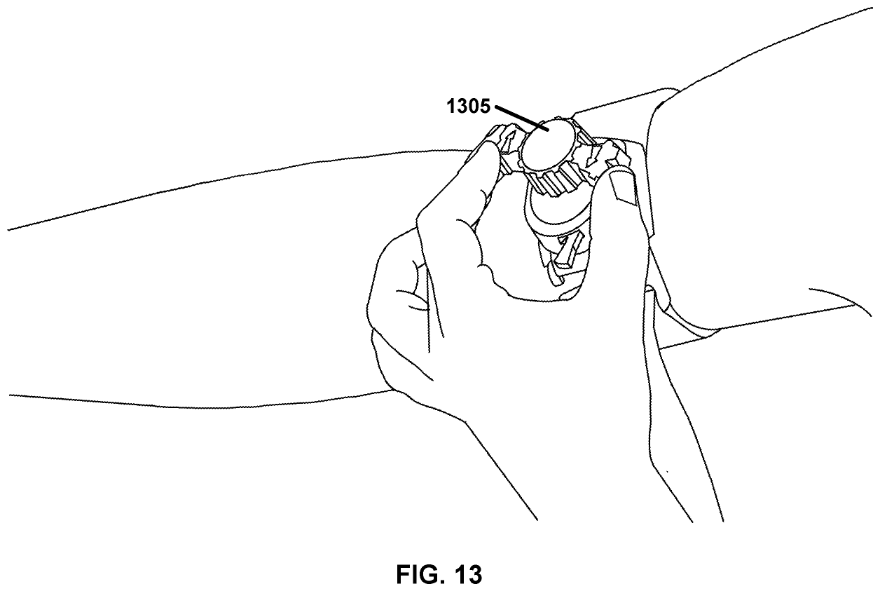

FIG. 13 illustrates the winding of an example knob of an example tightening mechanism 1305 of an example tourniquet consistent with disclosed embodiments. The tourniquet may comprise a tunnel strap and a tunnel strap quick release connector. The tunnel strap quick release connector may be configured to prevent the tunnel strap from sliding through the tunnel strap quick release connector once the tunnel strap has been tightened around an extremity of a wearer. After tightening, the tunnel strap may be secured through employment of a hook and loop fastener. The tourniquet may be tightened further as needed to occlude blood flow in the extremity through employment of the knob of the tightening mechanism 1305.

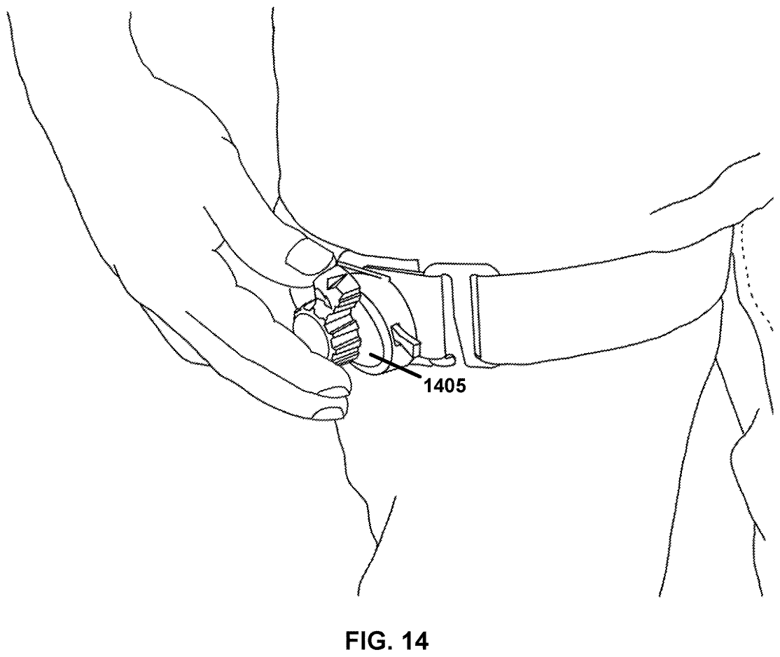

FIG. 14 illustrates the winding of an example knob of an example tightening mechanism 1405 of an example tourniquet applied over example clothing consistent with disclosed embodiments. The tourniquet may comprise a tunnel strap and a tunnel strap quick release connector. The tunnel strap quick release connector may be configured to prevent the tunnel strap from sliding through the tunnel strap quick release connector once the tunnel strap has been tightened around an extremity of a wearer wearing the clothing. After tightening, the tunnel strap may be secured through employment of a hook and loop fastener. The tourniquet may be tightened further as needed to occlude blood flow in the extremity through employment of the knob of the tightening mechanism 1405.

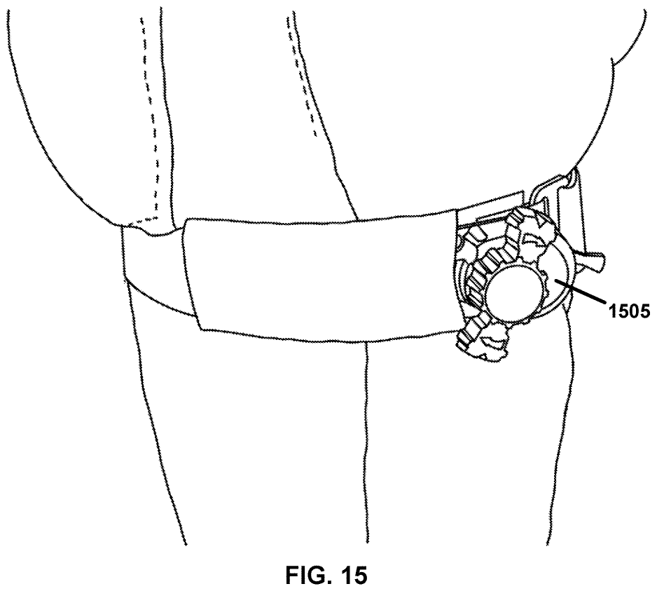

FIG. 15 illustrates an example tourniquet applied over example clothing consistent with disclosed embodiments. The tourniquet may comprise a tightening mechanism 1505. The tourniquet may comprise a tunnel strap and a tunnel strap quick release connector. The tunnel strap quick release connector may be configured to prevent the tunnel strap from sliding through the tunnel strap quick release connector once the tunnel strap has been tightened around an extremity of a wearer wearing the clothing. After tightening, the tunnel strap may be secured through employment of a hook and loop fastener. The tourniquet may be configured to occlude blood flow in the extremity once the tightening mechanism 1505 has been tightened.

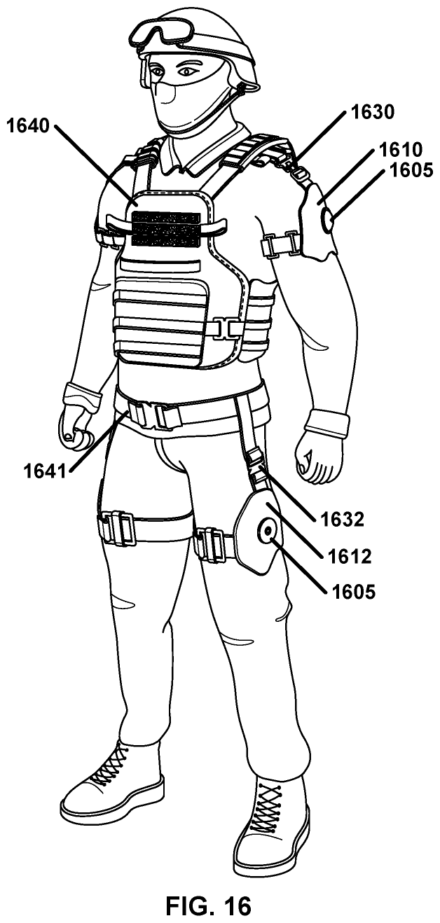

FIG. 16 illustrates a first view of example wearable devices (1610, 1612) attached to example wearable items (1640, 1641) consistent with disclosed embodiments. A first wearable device 1610 may comprise a tightening mechanism 1605. The first wearable device 1610 may be attached to a first wearable item such as body armor 1640 through employment of a first wearable item quick release connector 1630. A second wearable device 1612 may comprise a tightening mechanism 1605. The second wearable device 1612 may be attached to a second wearable item such as a belt 1641 through employment of a second wearable item quick release connector 1632.

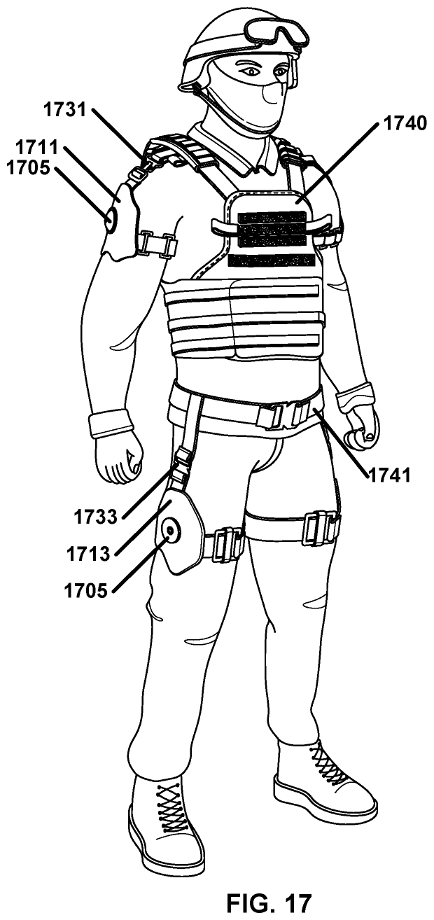

FIG. 17 illustrates a second view of example wearable devices (1711, 1713) attached to example wearable items (1740, 1741) consistent with disclosed embodiments. A first wearable device 1711 may comprise a tightening mechanism 1705. The first wearable device 1711 may be attached to a first wearable item such as body armor 1740 through employment of a first wearable item quick release connector 1731. A second wearable device 1713 may comprise a tightening mechanism 1705. The second wearable device 1713 may be attached to a second wearable item such as a belt 1741 through employment of a second wearable item quick release connector 1733.

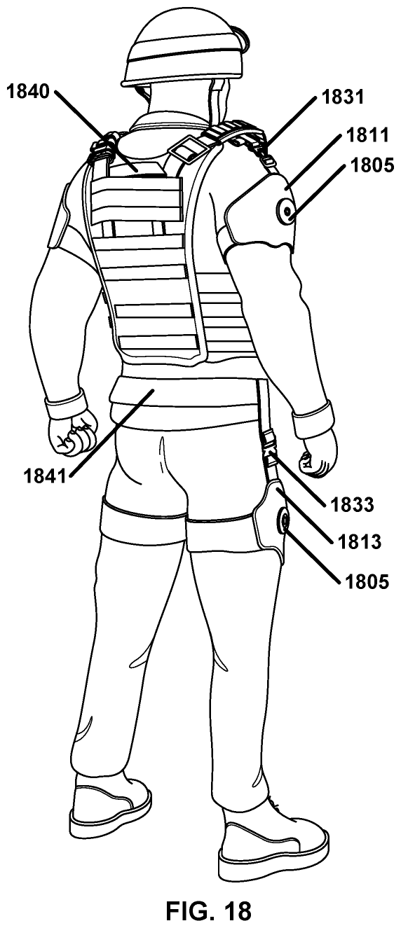

FIG. 18 illustrates a third view of example wearable devices (1811, 1813) attached to example wearable items (1840, 1841) consistent with disclosed embodiments. A first wearable device 1811 may comprise a tightening mechanism 1805. The first wearable device 1811 may be attached to a first wearable item such as body armor 1840 through employment of a first wearable item quick release connector 1831. A second wearable device 1813 may comprise a tightening mechanism 1805. The second wearable device 1813 may be attached to a second wearable item such as a belt 1841 through employment of a second wearable item quick release connector 1833.

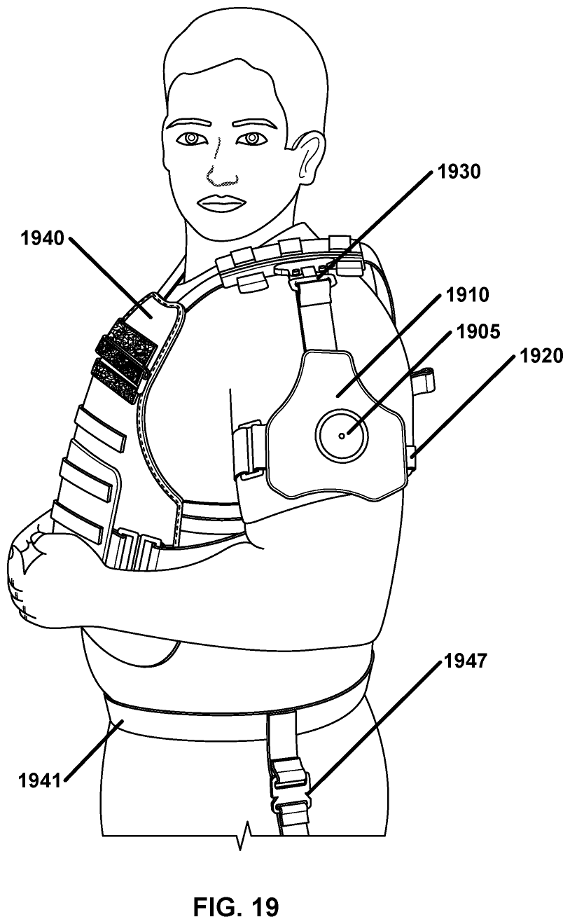

FIG. 19 illustrates a first view of an example wearable device 1910 and example wearable items (1940,1941) consistent with disclosed embodiments. The wearable device 1910 may be attached to wearable item 1940 through employment of a wearable item quick release connector 1930. A first wearable item 1940 may comprise body armor. A second wearable item 1941 may comprise a belt. The second wearable item 1941 may comprise a second wearable item connector 1947. The wearable device 1910 may comprise a tourniquet. The tourniquet may comprise a tightening mechanism 1905. The tourniquet may comprise a tunnel strap 1920.

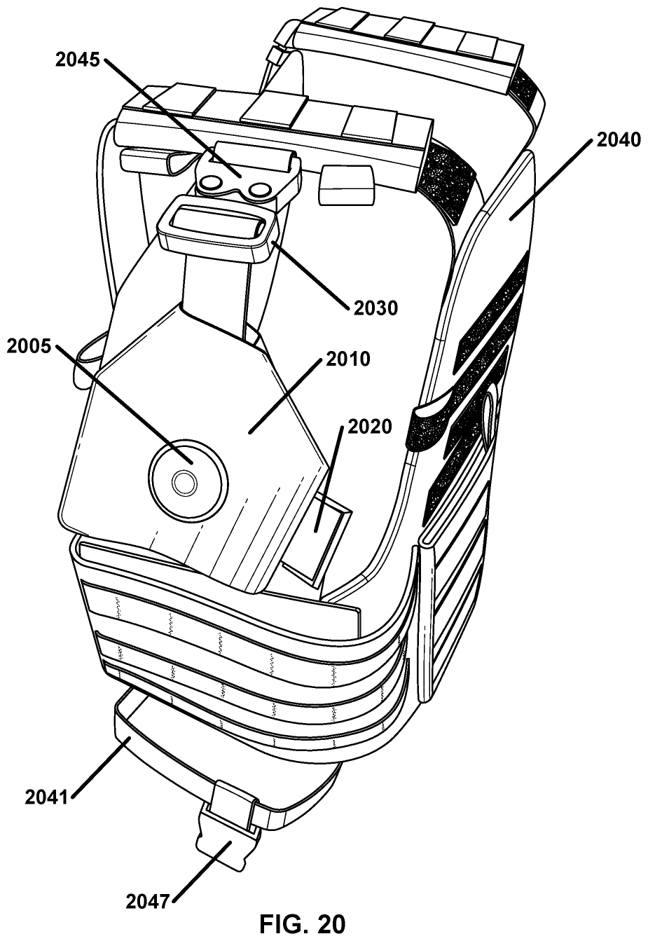

FIG. 20 illustrates a second view of an example wearable device 2010 and example wearable items (2040,2041) consistent with disclosed embodiments. The wearable device 2010 may be attached to wearable item 2040 through employment of an example wearable item connector 2045 and an example wearable item quick release connector 2030. The wearable item 2040 may comprise body armor. The wearable item 2040 may comprise the wearable item connector 2045. The wearable item connector 2045 may be configured to connect to the wearable item quick release connector 2030. The wearable device 2010 may comprise a tourniquet. The tourniquet may comprise a tightening mechanism 2005. The tourniquet may comprise a tunnel strap 2020. A second wearable item 2041 may comprise a belt. The second wearable item 2041 may comprise a second wearable item connector 2047. The second wearable item connector 2047 may be configured to receive a wearable item quick release connector of a second wearable device.

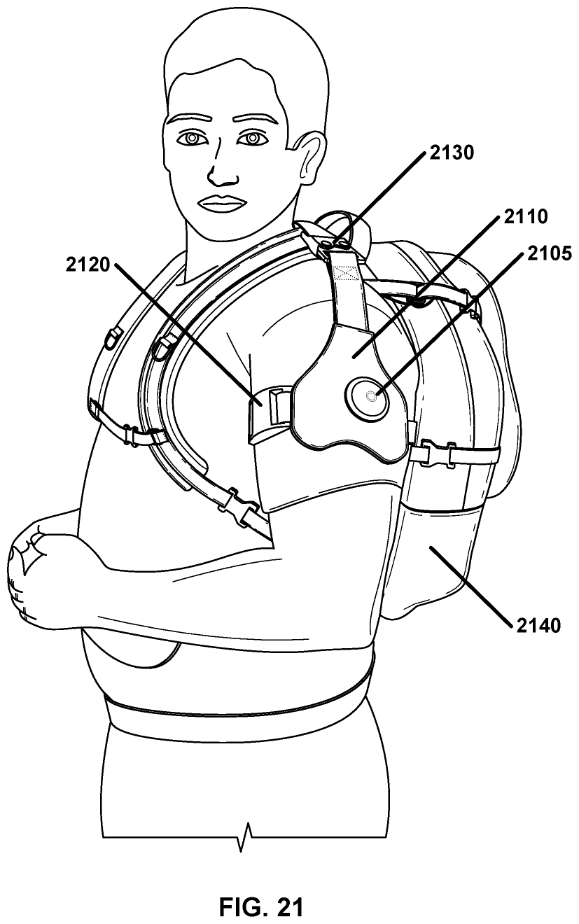

FIG. 21 illustrates an example wearable device 2110 attached to an example wearable item 2140 consistent with disclosed embodiments. The wearable device 2110 may be attached to the wearable item 2140 through employment of a wearable item quick release connector 2130. The wearable item 2140 may comprise a backpack. The wearable device 2110 may comprise a tourniquet. The tourniquet may comprise a tightening mechanism 2105. The Tourniquet may comprise a tunnel strap 2120.

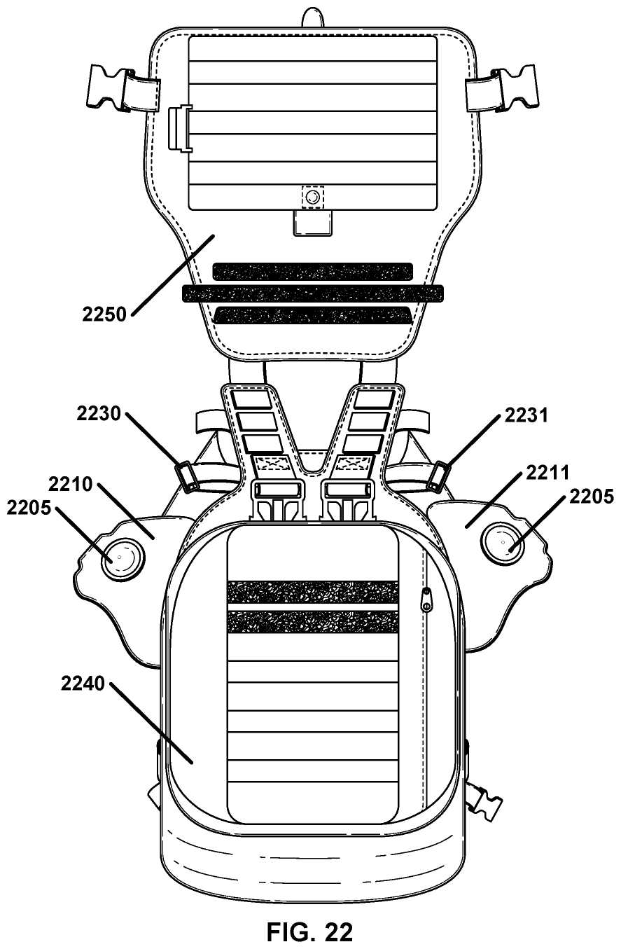

FIG. 22 illustrates a first view of example wearable devices (2210, 2211) attached to an example backpack 2240 with an example deployable front ballistic plate carrier 2250 consistent with disclosed embodiments. The deployable front ballistic plate carrier 2250 may be configured to be stowed in the backpack 2240 until needed. The deployable front ballistic plate carrier 2250 may be configured to be removably connected to the backpack 2240. Each of the wearable devices (2210, 2211) may be attached to the backpack 2240 through employment of a wearable item quick release connector (2230, 2231). Each of the wearable devices (2210, 2211) may comprise a tourniquet. Each tourniquet may comprise a tightening mechanism 2205.

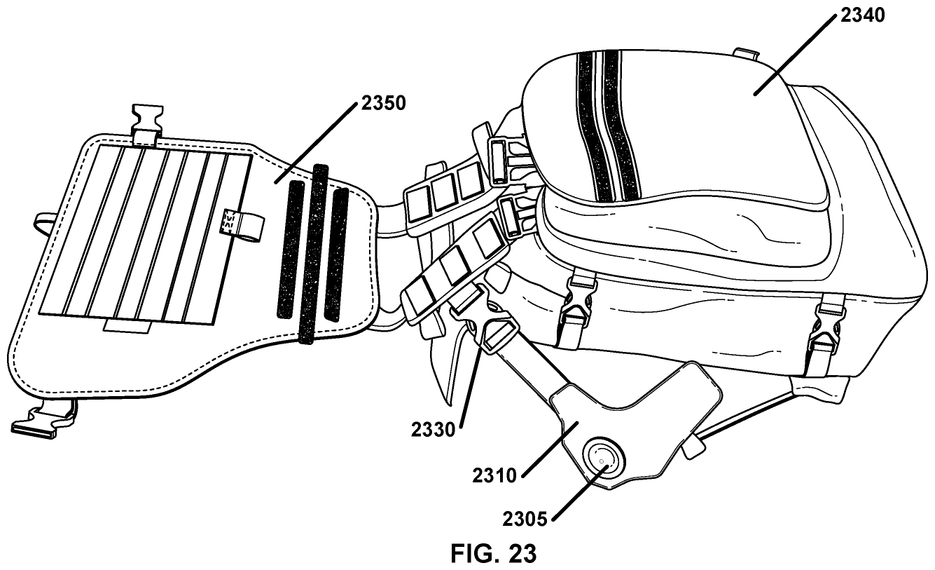

FIG. 23 illustrates a second view of example an wearable device 2310 attached to an example backpack 2340 with an example deployable front ballistic plate carrier 2350 consistent with disclosed embodiments. The deployable front ballistic plate carrier 2350 may be configured to be stowed in the backpack 2340 until needed. The deployable front ballistic plate carrier 2350 may be configured to be removably connected to the backpack 2340. The wearable device 2310 may be attached to the backpack 2340 through employment of a wearable item quick release connector 2330. The wearable device 2310 may comprise a tourniquet. The tourniquet may comprise a tightening mechanism 2305.

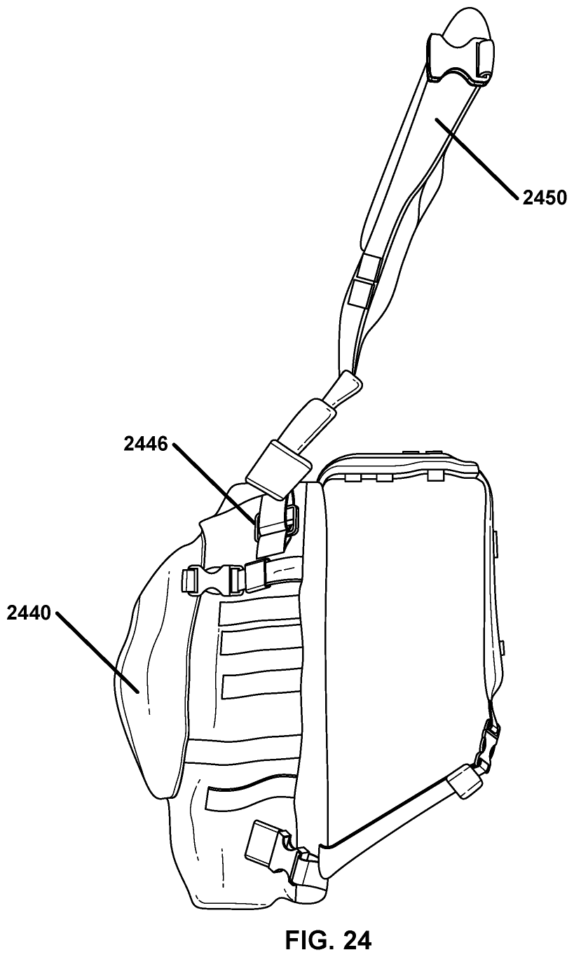

FIG. 24 illustrates an example backpack 2440 with an example deployable front ballistic plate carrier 2450 consistent with disclosed embodiments. The backpack 2440 may comprise a wearable item connector 2446. The wearable item connector 2446 may be configured to receive a wearable item quick release connector of a wearable device.

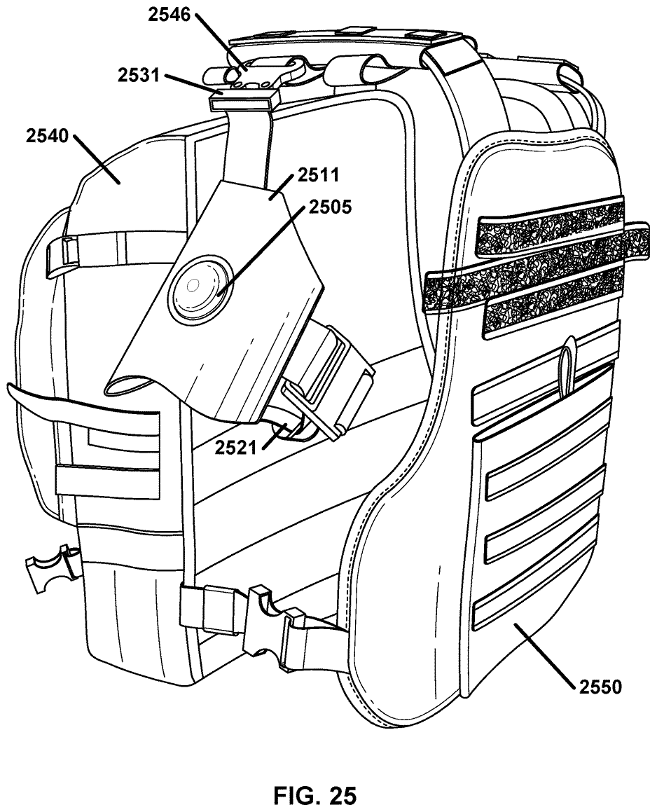

FIG. 25 illustrates an example wearable device 2511 attached to an example backpack 2540 with an example deployable front ballistic plate carrier 2550 in a deployed position consistent with disclosed embodiments. The example wearable device 2511 may be attached to the backpack 2540 through employment of a wearable item quick release connector 2531. The backpack 2540 may comprise a wearable item connector 2546. The wearable item connector 2546 may be configured to connect to the wearable item quick release connector 2531. The wearable device 2511 may comprise a tourniquet. The tourniquet may comprise a tightening mechanism 2505. The tourniquet may comprise a tunnel strap 2521.

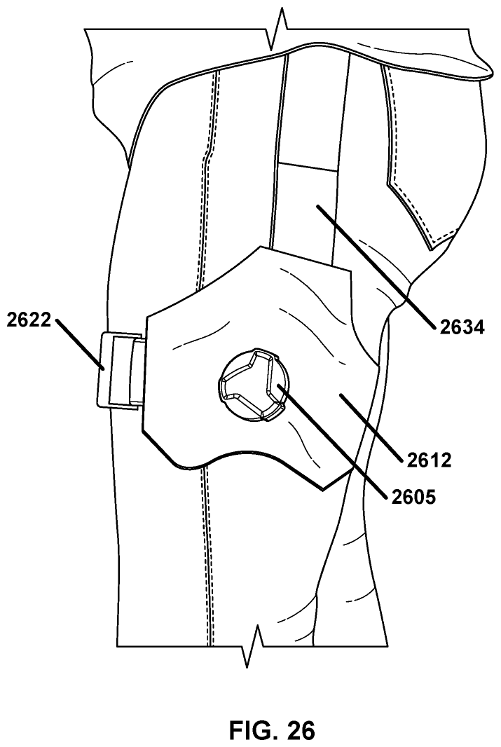

FIG. 26 illustrates an example wearable device 2612 attached to an example wearable item (for example, 1641, not shown) consistent with disclosed embodiments. The wearable device 2612 may comprise a wearable item strap 2634. The wearable item strap 2634 may be configured to removably connect to the wearable item (for example, 1641). The wearable item strap 2634 may comprise a hook and loop fastener. The wearable device 2612 may comprise a tourniquet. The tourniquet may comprise a tightening mechanism 2605. The tourniquet may comprise a tunnel strap 2622.

According to an embodiment, a tightening mechanism may comprise a spool. The spool may be configured to accept a portion of at least one lace. The tightening mechanism may comprise a ratchet wheel. The ratchet wheel may be operably connected to the spool. The ratchet wheel may include a plurality of ratchet teeth. The tightening mechanism may comprise a knob. The knob may be operably connected to the ratchet wheel. The knob may be configured for manually winding a portion of the at least one lace onto the spool in a winding direction. The tightening mechanism may comprise a first pawl arm. The first pawl arm may comprise a first pawl. The first pawl may be configured to engage with each of the plurality of ratchet teeth to prevent the spool from moving in a direction opposite to the winding direction. The tightening mechanism may comprise a second pawl arm. The second pawl arm may comprise a second pawl. The second pawl may be configured to engage with each of the plurality of ratchet teeth to prevent the spool from moving in the direction opposite to the winding direction. The tightening mechanism may comprise a spring. The spring may be configured to bias the first pawl and the second pawl towards the ratchet wheel.

According to an embodiment, a knob may comprise a center portion. The center portion may comprise a circular shape. The knob may comprise two extensions. Each of the two extensions may extend radially outward from the center portion.

According to an embodiment, a center portion may comprise a plurality of protrusions.

According to an embodiment, two extensions may each comprise a plurality of protrusions.

According to an embodiment, a spring may comprise a torsion spring. A first pawl arm may comprise a first boss. The first boss may be configured to restrain a first end of the torsion spring.

According to an embodiment, a spring may comprise a torsion spring. A second pawl arm may comprise a second boss. The second boss may be configured to restrain a second end of the torsion spring.

According to an embodiment, a tightening mechanism may comprise a toggle arm.

The toggle arm may be configured to alternate disengagement of a first pawl and a second pawl from a ratchet wheel.

According to an embodiment, a toggle arm may comprise a release lever. When the release lever is actuated in a first release direction, the release lever may be configured to release a first pawl from a ratchet wheel. When the release lever is actuated in a second release direction, the release lever may be configured to release a second pawl from the ratchet wheel.

According to an embodiment, a release lever may comprise a plurality of protrusions.

According to an embodiment, a tightening mechanism may comprise a first pawl arm pivot pin. A first pawl arm may be configured to pivot about the first pawl arm pivot pin.

According to an embodiment, a tightening mechanism may comprise a second pawl arm pivot pin. A second pawl arm may be configured to pivot about the second pawl arm pivot pin.

According to an embodiment, a tightening mechanism may comprise a toggle arm pivot pin. A toggle arm may be configured to pivot about the toggle arm pivot pin.

According to an embodiment, a tightening mechanism may comprise a lace anchor pin. The lace anchor pin may be configured to anchor at least one lace to the tightening mechanism.

According to an embodiment, a tightening mechanism may comprise a lower housing.

According to an embodiment, a lower housing may be configured to be fastened to an article.

According to an embodiment, an article may comprise a base of a tourniquet.

According to an embodiment, a tightening mechanism may comprise an upper housing. The upper housing may be configured to connect to a lower housing.

According to an embodiment, an upper housing may be configured to be fastened to a lower housing.

According to an embodiment, an upper housing may be connected to a lower housing. The upper housing and the lower housing together may be configured to house a spool, a ratchet wheel, a first pawl arm, a second pawl arm, and a spring.

According to an embodiment, a tightening mechanism may comprise an upper housing and a lower housing. The upper housing may be connected to the lower housing. The upper housing and the lower housing together may be configured to house a toggle arm.

According to an embodiment, an upper housing may define a release lever aperture.

According to an embodiment, an upper housing may define a lace path.

According to an embodiment, a lace path may include a plurality of lace apertures.

According to an embodiment, a tightening mechanism may comprise a knob bolt. The knob bolt may be configured to fasten a spool and a ratchet wheel to a knob.

According to an embodiment, a wearable device may comprise a tourniquet. The tourniquet may comprise a base. The tourniquet may comprise a tightening mechanism. The tightening mechanism may be connected to the base. The tightening mechanism may comprise a spool. The spool may be configured to accept a first portion of a lace. The tightening mechanism may comprise a knob. The knob may be configured for manually winding the first portion of the lace onto the spool. The tightening mechanism may comprise an anchor point. The anchor point may be configured to anchor a second portion of the lace. The tourniquet may comprise a tunnel strap. The tourniquet may comprise a tunnel sled. The tunnel sled may be connected to a first end of the tunnel strap. The tunnel sled may comprise a first lace guide. The first lace guide may be configured to guide the lace from the anchor point directly to the spool. The tunnel sled may comprise a second lace guide. The second lace guide may be configured to guide the lace from the anchor point directly to the spool.

The wearable device may comprise a wearable item quick release connector. The wearable item quick release connector may be connected to the tourniquet. The wearable item quick release connector may be configured to removably connect to a wearable item.

According to an embodiment, a wearable item may comprise at least one of a ballistic plate carrier, a backpack, a belt, or a harness.

According to an embodiment, a wearable item quick release connector may be magnetic.

According to an embodiment, a wearable device may comprise an outer cuff. The outer cuff may be configured to cover at least a portion of a tourniquet.

According to an embodiment, a tightening mechanism may comprise a housing. The housing may be configured to house a spool, a first portion of a lace, and a second portion of the lace.

According to an embodiment, a housing may be fastened to a base.

According to an embodiment, a tourniquet may comprise a tunnel strap quick release connector. The tunnel strap quick release connector may be connected to a second portion of a tunnel strap. The tunnel strap quick release connector may be configured to removably connect to a second end of a base.

According to an embodiment, a tunnel strap quick release connector may comprise a G hook.

According to an embodiment, a tunnel strap quick release connector may be magnetic.

According to an embodiment, a base may comprise a base loop. The base loop may be configured to receive a tunnel strap quick release connector.

According to an embodiment, a base may comprise a first base loop. The first base loop may be configured to receive a tunnel strap quick release connector. The base may comprise a second base loop. The second base loop may be configured to receive the tunnel strap quick release connector. The first base loop may be configured for larger limbs. The second base loop may be configured for smaller limbs.

According to an embodiment, a tourniquet may comprise a base. The tourniquet may comprise a tightening mechanism. The tightening mechanism may be connected to the base. The tightening mechanism may comprise a spool. The spool may be configured to accept a first portion of a lace. The tightening mechanism may comprise a knob. The knob may be configured for manually winding the first portion of the lace onto the spool. The tightening mechanism may comprise an anchor point. The anchor point may be configured to anchor a second portion of the lace. The tourniquet may comprise a tunnel strap. The tourniquet may comprise a tunnel sled. The tunnel sled may be connected to a first end of the tunnel strap.

The tunnel sled may comprise a first lace guide. The first lace guide may be configured to guide the lace from the anchor point directly to the spool. The tunnel sled may comprise a second lace guide. The second lace guide may be configured to guide the lace from the anchor point directly to the spool.

According to an embodiment, a tourniquet may comprise an outer cuff. The outer cuff may be configured to cover at least a portion of a base.

According to an embodiment, a tightening mechanism may comprise a release lever.

According to an embodiment, a tightening mechanism may comprise a housing. The housing may be configured to house a spool, a first portion of a lace, and a second portion of the lace.

According to an embodiment, a housing may be fastened to a base.

According to an embodiment, a tourniquet may comprise a tunnel strap quick release connector. The tunnel strap quick release connector may be connected to a second portion of a tunnel strap. The tunnel strap quick release connector may be configured to removably connect to a second end of a base.

According to an embodiment, a tunnel strap quick release connector may comprise a G hook

According to an embodiment, a tunnel strap quick release connector may be magnetic.

According to an embodiment, a base may comprise a base loop. The base loop may be configured to receive a tunnel strap quick release connector.

According to an embodiment, a base may comprise a first base loop. The first base loop may be configured to receive a tunnel strap quick release connector. The base may comprise a second base loop. The second base loop may be configured to receive the tunnel strap quick release connector. The first base loop may be configured for larger limbs. The second base loop may be configured for smaller limbs.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In this specification, “a” and “an” and similar phrases are to be interpreted as “at least one” and “one or more.” References to “a”, “an”, and “one” are not to be interpreted as “only one”. In this specification, the term “may” is to be interpreted as “may, for example.” In other words, the term “may” is indicative that the phrase following the term “may” is an example of one of a multitude of suitable possibilities that may, or may not, be employed to one or more of the various embodiments. In this specification, the phrase “based on” is indicative that the phrase following the term “based on” is an example of one of a multitude of suitable possibilities that may, or may not, be employed to one or more of the various embodiments. References to “an” embodiment in this disclosure are not necessarily to the same embodiment.

While various embodiments have been described above, it should be understood that they have been presented by way of example, and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. Thus, the present embodiments should not be limited by any of the above described example embodiments.

In this specification, various embodiments are disclosed. Limitations, features, and/or elements from the disclosed example embodiments may be combined to create further embodiments within the scope of the disclosure. Moreover, the scope includes any and all embodiments having equivalent elements, modifications, omissions, adaptations, or alterations based on the present disclosure. Further, aspects of the disclosed methods can be modified in any manner, including by reordering aspects, or inserting or deleting aspects.

In addition, it should be understood that any figures that highlight any functionality and/or advantages, are presented for example purposes only. The disclosed architecture is sufficiently flexible and configurable, such that it may be utilized in ways other than that shown. For example, the blocks presented in any flowchart may be re-ordered or only optionally used in some embodiments.

Furthermore, many features presented above are described as being optional through the use of “may” or the use of parentheses. For the sake of brevity and legibility, the present disclosure does not explicitly recite each and every permutation that may be obtained by choosing from the set of optional features. However, the present disclosure is to be interpreted as explicitly disclosing all such permutations. For example, a system described as having three optional features may be embodied in seven different ways, namely with just one of the three possible features, with any two of the three possible features, or with all three of the three possible features.

Further, the purpose of the Abstract of the Disclosure is to enable Patent Offices, and the public generally, and especially the scientists, engineers and practitioners in the art who are not familiar with patent or legal terms or phraseology, to determine quickly from a cursory inspection the nature and essence of the technical disclosure of the application. The Abstract of the Disclosure is not intended to be limiting as to the scope in any way.

Finally, it is the applicant's intent that only claims that include the express language “means for” or “step for” be interpreted under 35 U.S.C. 112. Claims that do not expressly include the phrase “means for” or “step for” are not to be interpreted under 35 U.S.C. 112.

1. A tightening mechanism comprising: a) a spool being configured to accept a portion of at least one lace; b) a ratchet wheel operably connected to the spool, the ratchet wheel including a plurality of ratchet teeth; c) a knob operably connected to the ratchet wheel, the knob being configured for manually winding a portion of the at least one lace onto the spool in a winding direction; d) a first pawl arm comprising a first pawl, the first pawl being configured to engage with each of the plurality of ratchet teeth to prevent the spool from moving in a direction opposite to the winding direction; e) a second pawl arm comprising a second pawl, the second pawl being configured to engage with each of the plurality of ratchet teeth to prevent the spool from moving in the direction opposite to the winding direction; and f) a spring being configured to bias the first pawl and the second pawl towards the ratchet wheel.

2. The tightening mechanism according to claim 1, wherein the knob comprises a center portion having a circular shape, and two extensions, each of the two extensions extending radially outward from the center portion, wherein the center portion and the two extensions each comprise a plurality of protrusions.

3. (canceled)

4. The tightening mechanism according to claim 1, wherein the spring comprises a torsion spring, and the first pawl arm comprises a first boss being configured to restrain a first end of the torsion spring.

5. The tightening mechanism according to claim 1, wherein the spring comprises a torsion spring, and the second pawl arm comprises a second boss being configured to restrain a second end of the torsion spring.

6. The tightening mechanism according to claim 1, further comprising a toggle arm being configured to alternate disengagement of the first pawl and the second pawl from the ratchet wheel.

7. The tightening mechanism according to claim 6, wherein the toggle arm comprises a release lever that when actuated in a first release direction, is configured to release the first pawl from the ratchet wheel, and when actuated in a second release direction, is configured to release the second pawl from the ratchet wheel.

8. The tightening mechanism according to claim 7, wherein the release lever comprises a plurality of protrusions.

9. The tightening mechanism according to claim 1, further comprising a first pawl arm pivot pin, the first pawl arm being configured to pivot about the first pawl arm pivot pin, and further comprising a second pawl arm pivot pin, the second pawl arm being configured to pivot about the second pawl arm pivot pin.

10. (canceled)

11. The tightening mechanism according to claim 6, further comprising a toggle arm pivot pin, the toggle arm being configured to pivot about the toggle arm pivot pin.

12. The tightening mechanism according to claim 1, further comprising a lace anchor pin, the lace anchor pin being configured to anchor the at least one lace to the tightening mechanism.

13. The tightening mechanism according to claim 1, further comprising a lower housing, wherein the lower housing is configured to be fastened to an article, and wherein the article comprises a base tourniquet.

14. (canceled)

15. (canceled)

16. The tightening mechanism according to claim 13, further comprising an upper housing, the upper housing being configured to connect to the lower housing, wherein the upper housing is configured to be fastened to the lower housing.

17. (canceled)

18. The tightening mechanism according to claim 16, wherein the upper housing is connected to the lower housing, the upper housing and the lower housing together being configured to house: a) the spool; b) the ratchet wheel; c) the first pawl arm; d) the second pawl arm; and e) the spring.

19. The tightening mechanism according to claim 6, further comprising an upper housing and a lower housing, the upper housing connected to the lower housing, the upper housing and the lower housing together being configured to house the toggle arm.

20. The tightening mechanism according to claim 16, wherein the upper housing defines a release lever aperture.

21. The tightening mechanism according to claim 16, wherein the upper housing defines a lace path.

22. The tightening mechanism according to claim 21, wherein the lace path includes a plurality of lace apertures.

23. The tightening mechanism according to claim 1, further comprising a knob bolt being configured to fasten the spool and the ratchet wheel to the knob.

24. A wearable device comprising: a) a tourniquet comprising: i) a base; ii) a tightening mechanism connected to the base, the tightening mechanism comprising:
A) a spool being configured to accept a first portion of a lace;
B) a knob being configured for manually winding the first portion of the lace onto the spool; and
C) an anchor point being configured to anchor a second portion of the lace; iii) a tunnel strap; and iv) a tunnel sled connected to a first end of the tunnel strap, the tunnel sled comprising:
A) a first lace guide being configured to guide the lace from the anchor point directly to the spool; and
B) a second lace guide being configured to guide the lace from the anchor point directly to the spool; and b) a wearable item quick release connector connected to the tourniquet and being configured to removably connect to a wearable item.

25-34. (canceled)

35. A tourniquet comprising: a) a base; b) a tightening mechanism connected to the base, the tightening mechanism comprising: i) a spool being configured to accept a first portion of a lace; ii) a knob being configured for manually winding the first portion of the lace onto the spool; and iii) an anchor point being configured to anchor a second portion of the lace; c) a tunnel strap; and d) a tunnel sled connected to a first end of the tunnel strap, the tunnel sled comprising: i) a first lace guide being configured to guide the lace from the anchor point directly to the spool; and ii) a second lace guide being configured to guide the lace from the anchor point directly to the spool.

36-44. (canceled)
